(19) United States
(12) Patent Application Publication
(10) Pub. No.: US 2026/0070869 A1
(43) Pub. Date: Mar. 12, 2026

(54) PROCESS FOR PREPARING CARBOXYLIC ACID ESTERS OF HYDROXYBUTANOATES

(71) Applicant: KetoLipix Therapeutics GmbH, Hamburg (DE)

(72) Inventors: Dirk LOCHMANN, Hamburg (DE); Sebastian REYER, Hamburg (DE); Michael STEHR, Hamburg (DE)

(21) Appl. No.: 19/328,682

(22) Filed: Sep. 15, 2025

Related U.S. Application Data
(62) Division of application No. 18/004,748.

(30) Foreign Application Priority Data
Jul. 13, 2020 (WO) PCT/EP2020/069707

(51) Int. Cl. C07C 69/675; A23L 33/10; A61P 3/00

(52) U.S. Cl. CPC C07C 69/675; A23L 33/10; A61P 3/00

(57) ABSTRACT

The invention relates to a method for producing carboxylic acid esters of 3-hydroxybutanoates (i.e. carboxylic acid esters of 4-oxo-2-butanols), especially carboxylic acid esters of 4-oxo-4-(C₁-C₅-alkoxy)-2-butanol or of 4-oxo-4-(hydroxy-C₃-C₅-alkoxy)-2-butanol, as well as the products thus obtained and their use.

### CROSS-REFERENCES TO RELATED APPLICATIONS

This is a divisional application that claims priority to and the benefit of co-pending U.S. patent application Ser. No. 18/004,748, filed on Jan. 9, 2023, which is a National Stage filing of International Application PCT/EP 2020/074890 filed Sep. 7, 2020, entitled “Method for Preparing Carboxylic Acid Esters of Hydroxybutanoates” claiming priority to PCT/EP 2020/069707 filed Jul. 13, 2020. The subject application claims priority to PCT/EP 2020/074890 and PCT/EP 2020/069707 and incorporates all by reference herein, in their entirety.

### BACKGROUND OF THE INVENTION

The present invention relates to the field of keto bodies and related metabolism and the therapy of related diseases.

Especially, the present invention relates to a method for producing carboxylic acid esters of 3-hydroxybutanoate, as well as the reaction products thus obtainable or thus prepared (i.e. carboxylic acid esters of 3-hydroxybutanoate) and their use, especially in pharmaceutical compositions, such as drugs or medicaments, or in food and/or food products, as well as their further applications or uses.

Furthermore, the present invention relates to pharmaceutical compositions, especially drugs or medicaments, comprising the reaction products (i.e. carboxylic acid esters of 3-hydroxybutanoate) obtainable or produced according to the inventive method, as well as their applications or uses.

Finally, the present invention relates to food and/or food products, especially food supplements, functional foods, novel foods, food additives, food supplements, dietary foods, power snacks, appetite suppressants and strength and/or endurance sports supplements, which comprise the reaction products (i.e. carboxylic acid esters of 3-hydroxybutanoate) obtainable or produced according to the inventive method, as well as their applications or uses.

In the human energy metabolism, glucose is the short-term available energy carrier, which is metabolized into energy in the mitochondria by releasing water and carbon dioxide. The glycogen stores of the liver are already emptied during the sleep period during the night. However, especially the human central nervous system (CNS) and the heart require a permanent energy supply.

The physiological alternative to glucose, which is mainly available to the central nervous system, are the so-called keto bodies (synonymously also called ketone bodies).

The term keto body is especially a collective term for three compounds, which are formed mainly in catabolic metabolic states (such as hunger, reduction diets or low-carbohydrate diets) and may lead to ketosis. The term keto bodies includes especially the three compounds acetoacetate (synonymously also referred to as acetacetate) and acetone as well as 3-hydroxybutyric acid (hereinafter also synonymously referred to as beta-hydroxybutyric acid or BHB or 3-BHB) or its salt (i.e. 3-hydroxybutyrate or beta-hydroxybutyrate), the latter being the most important of the three aforementioned compounds. 3-Hydroxybutyric acid or its salt occurs physiologically as the (R)-enantiomer, i.e. as (R)-3-hydroxybutyric acid (synonymously also called (3R)-3-hydroxybutyric acid to emphasize the center of chirality in the 3-position) or its salt.

These keto bodies are also provided physiologically in large amounts from lipids stored in the body by lipolysis during fasting or starvation and replace the energy source glucose almost completely.

The keto bodies are formed in the liver from acetyl coenzyme A (=acetyl-CoA), which originates from beta-oxidation; they represent a transportable form of the acetyl coenzyme A in the human body. However, in order to utilize the keto bodies, the brain and muscles must first adapt by expressing enzymes that are required to convert keto bodies back into acetyl coenzyme A. Especially in times of hunger, the keto bodies contribute a considerable amount to energy production. For example, after some time the brain is able to get by with only a third of the daily amount of glucose.

Physiologically, the keto bodies are synthesized from two molecules of activated acetic acid in the form of acetyl coenzyme A, the normal intermediate product of fatty acid degradation, which is extended using a further acetyl coenzyme A unit and the enzyme HMG-COA-synthase to the intermediate product 3-hydroxy-3-methyl-glutaryl-CoA (HMG-CoA), wherein finally the HMG-CoA-lyase cleaves off the acetoacetate. These three steps take place exclusively in the mitochondria of the liver (lynen cycle), wherein 3-hydroxybutyrate is finally formed in the cytosol by the D-beta-hydroxybutyrate dehydrogenase. HMG-CoA is also an end product of the degradation of the amino acid leucine, while acetoacetate is formed during the degradation of the amino acids phenylalanine and tyrosine.

Spontaneous decarboxylation turns acetoacetate into acetone; it can occasionally be perceived in the breath of diabetics and dieters. It cannot be further used by the body. However, the proportion of acetone in the keto bodies is small.

Acetoacetate is thus reductively converted into the physiologically relevant form of 3-hydroxybutyric acid or 3-hydroxybutyrate, but can also decompose into the physiologically unusable acetone with the release of carbon dioxide, which is detectable and olfactory perceptible in severe ketosis, a ketoacidosis (e.g. in diabetes mellitus type 1 patients without insulin substitution), in the urine and in the exhaled air.

3-Hydroxybutyric acid is currently used and marketed in the weight training sector as a sodium, magnesium or calcium salt.

However, 3-hydroxybutyric acid is not known or only in very small quantities to humans in evolutionary terms, since plants do not produce 3-hydroxybutyric acid and 3-hydroxybutyric acid in the animal organism only occurs in dead emaciated animals in ketosis, so that 3-hydroxybutyric acid causes nausea when administered orally. 3-Hydroxybutyric acid in the form of free acid and its salts also taste very bitter and can cause severe vomiting and nausea.

Moreover, patients, especially newborns, but also adults cannot permanently tolerate large amounts of salts of 3-hydroxybutyric acid, as these compounds can have a kidney-damaging effect.

In addition, the plasma half-life of 3-hydroxybutyric acid and its salts is so short that even if several grams are taken, the ketosis lasts only for about three to four hours, i.e. patients cannot benefit continuously from a therapy with 3-hydroxybutyric acid or its salts, especially at night. In case of metabolic diseases this can lead to life-threatening situations.

Therefore, in the case of the therapy of such metabolic diseases, so-called medium-chain triglycerides, so-called MCTs, are currently used for ketogenic therapy, i.e. the metabolic conversion of caproic, caprylic and capric acid (i.e. of saturated linear C₆-, C₈- and C₁₀-fatty acids) from the corresponding triglycerides is intended.

Basically, however, from a pharmaceutical and clinical point of view, 3-hydroxybutyric acid is a more effective pharmaceutical-pharmacological target molecule, which, according to the prior art, could in principle be used for the therapy of a large number of diseases, but cannot be used due to its lack of physiological compatibility (e.g. in diseases in connection with a malfunction of the energy metabolism, especially keto-body metabolism, or neurodegenerative diseases such as dementia, Alzheimer's disease, Parkinson's disease, etc., lipometabolic diseases etc.).

The following table illustrates purely exemplary, but by no means limiting, potential therapy options or possible indications for the active ingredient 3-hydroxybutyric acid.

| Indication | Therapeutic effect |
|---|---|
| Traumatic brain injury | Under BHB the apoptosis and necrosis rate of nerve cells decreases. |
| Stroke | Under BHB the apoptosis and necrosis rate of nerve cells decreases. |
| Refeeding syndrome | In case of anorexia, discontinuation of enteral or parenteral nutrition |
| | and after long periods of hunger, the consumption of starch or glucose |
| | can lead to death (see also WHO scheme peanut paste). BHB can be |
| | used here as a therapeutic agent to achieve normal food intake more |
| Appetite suppressant | BHB suppresses the feeling of hunger in the central nervous system |
| | (CNS). |
| Epilepsy | Conventional ketogenic diet to significantly reduce the frequency of |
| | seizures has extremely poor patient tolerance. BHB offers an |
| | immediately effective alternative here. |
| Alzheimer's disease, | Under BHB patients show better cognitive performance. BHB is also |
| dementia | effective in the prevention of neurodegenerative diseases. |
| Disorders of fatty acid | Compensation of a nutrient deficiency in case of defect in energy |
| oxidation (e.g. electron | metabolism. |
| transfer protein defect) | |

Therefore, it is desirable from a pharmaceutical and clinical point of view to be able to find effective precursors or metabolites which physiologically allow direct or indirect access to 3-hydroxybutyric acid or its salts, especially in the physiological metabolism of the human or animal body.

Consequently, the prior art has not lacked attempts to find physiologically suitable precursors or metabolites for 3-hydroxybutyric acid or its salts. So far, however, no efficient compounds have been found in the prior art. Also, access to such compounds is not or not readily possible according to the prior art.

The problem underlying the present invention is thus the provision of an efficient method for producing physiologically suitable or physiologically compatible precursors and/or metabolites of 3-hydroxybutyric acid (i.e. beta-hydroxybutyric acid or BHB or 3-BHB) or their salts.

Such method should especially make the respective BHB precursors and/or BHB metabolites accessible in an efficient way, especially in larger quantities and without significant amounts of toxic by-products.

### BRIEF SUMMARY OF THE INVENTION

In a completely surprising way, the applicant has now discovered that carboxylic acid esters of 3-hydroxybutanoate (3-hydroxybutyric acid ester) represent an efficient and physiologically effective or physiologically compatible precursor and/or metabolite for the keto body 3-hydroxybutyric acid or its salts and has in this context been able to find or develop an efficient method for producing these compounds, which allows direct and effective, especially economic as well as industrially feasible access to these compounds.

To solve the problem described hereinabove, the present invention therefore proposes—according to a first aspect of the present invention—a method for producing carboxylic acid esters of 3-hydroxybutanoate according to the teaching herein; further, especially special and/or advantageous embodiments of the inventive method are the subject-matter of the relevant claims.

Furthermore, the present invention relates—according to a second aspect of the present invention—to a reaction product obtainable according to the inventive method disclosed including a carboxylic acid ester of 3-hydroxybutanoate or a mixture of at least two carboxylic acid esters of 3-hydroxybutanoate.

Likewise, the present invention—according to a third aspect of the present invention—relates to a pharmaceutical composition, especially a drug or medicament; especially special and/or advantageous embodiments of this aspect of the invention.

Furthermore, the present invention—according to a fourth aspect of the present invention—relates to an inventive reaction product or an inventive carboxylic acid ester of 3-hydroxybutanoate or an inventive mixture of at least two carboxylic acid esters of 3-hydroxybutanoate for the prophylactic and/or therapeutic treatment or for use in the prophylactic and/or therapeutic treatment of diseases of the human or animal body.

Furthermore, the present invention—according to a fifth aspect of the present invention—relates to the use of an inventive reaction product or an inventive carboxylic acid ester of 3-hydroxybutanoate or an inventive mixture of at least two carboxylic acid esters of 3-hydroxybutanoate for the prophylactic and/or therapeutic treatment or for producing a medicament for the prophylactic and/or therapeutic treatment of diseases of the human or animal body.

Furthermore, the present invention—according to a sixth aspect of the present invention—relates to the use of an inventive reaction product or an inventive carboxylic acid ester of 3-hydroxybutanoate or an inventive mixture of at least two carboxylic acid esters of 3-hydroxybutanoate.

Furthermore, the present invention—according to a seventh aspect of the present invention—relates to a food and/or food product; further, especially special and/or advantageous embodiments of the food and/or food product are provided.

Finally, the present invention—according to an eighth aspect of the present invention-relates to the use of an inventive reaction product or an inventive carboxylic acid ester of 3-hydroxybutanoate or of an inventive mixture of at least two carboxylic acid esters of 3-hydroxybutanoate in a food and/or a food product.

It goes without saying that following features, embodiments, advantages and the like, which are subsequently listed below only with regard to one aspect of the invention for the purpose of avoiding repetition, naturally also apply accordingly to the other aspects of the invention, without this requiring a separate mention.

Furthermore, it goes without saying that individual aspects and embodiments of the present invention are also considered disclosed in any combination with other aspects and embodiments of the present invention and, especially, any combination of features and embodiments, as it results from back references of all patent claims, is also considered extensively disclosed with regard to all resulting combination possibilities.

With respect to all relative or percentage weight-based data provided below, especially relative quantity or weight data, it should further be noted that within the scope of the present invention these are to be selected by the person skilled in the art such that they always add up to 100% or 100% by weight, respectively, including all components or ingredients, especially as defined below; however, this is self-evident for the person skilled in the art.

In addition, the skilled person may, if necessary, deviate from the following range specifications without leaving the scope of the present invention.

Additionally, it applies that all values or parameters or the like specified in the following can be determined or identified in principle with standardized or explicitly specified determination methods or otherwise with the determination or measurement methods that are otherwise familiar to a person skilled in the art.

Having stated this, the present invention will be described in more detail hereinafter:

Thus, it is an object of the present invention—according to a first aspect of the present invention—to provide a method for producing carboxylic acid esters of 3-hydroxybutanoate, especially carboxylic acid esters of 4-oxo-4-(C₁-C₅-alkoxy)-2-butanol or of 4-oxo-4-(hydroxy-C₃-C₅-alkoxy)-2-butanol,
wherein at least one 3-hydroxybutanoate, especially a 4-oxo-4-(C₁-C₅-alkoxy)-2-butanol or a 4-oxo-4-(hydroxy-C₃-C₅-alkoxy)-2-butanol, of the general formula (I)

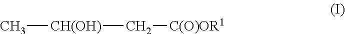

wherein, in general formula (I), the radical R¹ represents C₁-C₅-alkyl or hydroxy-C₃-C₅-alkyl, especially ethyl, butyl, pentyl, hydroxybutyl or hydroxypentyl, preferably ethyl, hydroxybutyl or hydroxypentyl, more preferably ethyl,
is reacted with at least one carboxylic acid (II), especially with at least one carboxylic acid comprising one or more carboxyl groups, preferably with at least one carboxylic acid comprising two or more carboxyl groups, especially in an esterification reaction and/or under esterification conditions,
so that, as a reaction product (III), one or more carboxylic acid esters of 3-hydroxybutanoate, especially one or more carboxylic acid esters of 4-oxo-4-(C₁-C₅-alkoxy)-2-butanol or of 4-oxo-4-(hydroxy-C₃-C₅-alkoxy)-2-butanol, are obtained.

In accordance with the present invention, there is thus provided a method for producing carboxylic acid esters of 3-hydroxybutanoate. 3-Hydroxybutanoate may also synonymously be referred to as 3-hydroxybutyric acid ester or alternatively as a 4-oxo-2-butanol.

Strictly speaking, the 3-hydroxybutanoate of the general formula (I) is either a (C₁-C₅-alkyl)-3-hydroxybutanoate (=3-hydroxybutyric acid (C₁-C₅-alkyl) ester), i.e. a C₁-C₅-alkyl ester of 3-hydroxybutyric acid, which may also be referred to synonymously as a 4-oxo-4-(C₁-C₅-alkoxy)-2-butanol, or else a (hydroxy-C₃-C₅-alkyl)-3-hydroxybutanoate (=3-hydroxybutyric acid (hydroxy-C₃-C₅-alkyl) ester), i.e. a hydroxy-C₃-C₅-alkyl ester of 3-hydroxybutyric acid, which can also be referred to synonymously as a 4-oxo-4-(hydroxy-C₃-C₅-alkoxy)-2-butanol.

In this context, the 3-hydroxybutanoates can be prepared by esterification of the free 3-hydroxybutyric acid with the corresponding alcohol (e.g. monoalcohol or diol). The synthesis of hydroxybutyl 3-hydroxybutanoate (i.e. 3-hydroxybutanoate of the general formula (I) with R¹=hydroxybutyl) is shown schematically below:

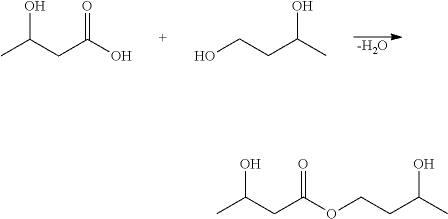

Furthermore, the synthesis of hydroxypentyl-3-hydroxybutanoate (i.e. 3-hydroxybutanoate of the general formula (I) with R¹=hydroxypentyl) is shown schematically below:

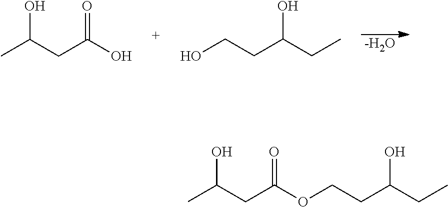

The preparation or synthesis of further 3-hydroxybutanoates proceeds analogously.

In the method according to the invention, the starting compound or reactant 3-hydroxybutanoate of the general formula (I) acts as an esterification alcohol via the hydroxyl group in the 3-position and reacts with the carboxyl group of the carboxylic acid (II), so that, as a reaction product (III), a corresponding carboxylic acid ester of 3-hydroxybutanoate, i.e. a corresponding carboxylic acid ester of 4-oxo-4-(C₁-C₅-alkoxy)-2-butanol or 4-oxo-4-(hydroxy-C₃-C₅-alkoxy)-2-butanol, is formed.

In the context of the present invention, the carboxylic acid (II) used is an organic carboxylic acid. That is, the carboxylic acid (II) is an organic compound having one or more carboxyl groups (—COOH), which has an acidic character.

Surprisingly, the applicant has found a way to provide 3-hydroxybutyric acid or its derivative in an organoleptically compatible form, while still allowing the 3-hydroxybutyric acid to be readily released, especially from the animal or human body.

In addition, the applicant has succeeded in providing the organoleptically compatible form of 3-hydroxybutyric acid in such a way that a retardation effect is present; i.e. the 3-hydroxybutyric acid is released continuously over a longer period of time, especially from the human or animal body.

Furthermore, the other cleavage products (i.e. the cleavage products which are released alongside to or in addition to 3-hydroxybutyric acid) can also be utilized by the body, or at least processed by the body. Especially, cleavage products are released which are reactants, products or intermediates of the citrate cycle or are derivatives or salts thereof which are formed by oxidation of a reactant, product or intermediate of the citrate cycle. Thus, the further cleavage products formed during the release of 3-hydroxybutyric acid can also be used as an energy source by the animal or human body. These cleavage products are typically the carboxylic acid (II) or its salts or derivatives.

When using carboxylic acids with at least two free carboxyl groups, it is possible that several 3-hydroxybutanoates react with one carboxylic acid (i.e. several 3-hydroxybutanoates are added to one carboxylic acid or one carboxylic acid is esterified with several 3-hydroxybutanoates), so that a high density of 3-hydroxybutanoates is present within one molecule and thus there is a high active ingredient density.

As stated above, the applicant has, quite surprisingly, discovered that the carboxylic acid esters of 3-hydroxybutanoate, especially carboxylic acid esters of 4-oxo-4-(C₁-C₅-alkoxy)-2-butanol or of 4-oxo-4-(hydroxy-C₃-C₅-alkoxy)-2-butanol, thus produced are efficient, since physiologically compatible precursors and/or metabolites of 3-hydroxybutyric acid or their salts, which can also be used in larger quantities in pharmaceutical or clinical applications because they are physiologically compatible.

The above-mentioned carboxylic acid esters of 3-hydroxybutanoate, especially carboxylic acid esters of 4-oxo-4-(C₁-C₅-alkoxy)-2-butanol or of 4-oxo-4-(hydroxy-C₃-C₅-alkoxy)-2-butanol, which are accessible for the first time in an efficient manner through the production method according to the invention, represent a physiologically and pharmacologically relevant alternative to free 3-hydroxybutyric acid or its salts.

The production of carboxylic acid esters of 3-hydroxybutanoate, especially carboxylic acid esters of 4-oxo-4-(C₁-C₅-alkoxy)-2-butanol or of 4-oxo-4-(hydroxy-C₃-C₅-alkoxy)-2-butanol, by means of conventional organic synthesis is complex and costly, since 3-hydroxybutyric acid as well as its salts and esters have an increased tendency to polymerize and to undergo other undesirable side reactions (e.g. dehydration, decomposition, etc.). Within the scope of the present invention, it was possible for the first time to provide an efficiently working production method with which carboxylic acid esters of 3-hydroxybutanoate, especially carboxylic acid esters of 4-oxo-4-(C₁-C₅-alkoxy)-2-butanol or of 4-oxo-4-(hydroxy-C₃-C₅-alkoxy)-2-butanol, can be produced at least essentially without undesired side reactions, especially in a single step.

The inventive method thus makes it possible for the first time to provide non-toxic carboxylic acid esters of 3-hydroxybutanoate, especially carboxylic acid esters of 4-oxo-4-(C₁-C₅-alkoxy)-2-butanol or of 4-oxo-4-(hydroxy-C₃-C₅-alkoxy)-2-butanol, from known, commercially available and above all physiologically harmless components or reactants (starting compounds). The resulting carboxylic acid esters of 3-hydroxybutanoate, especially carboxylic acid esters of 4-oxo-4-(C₁-C₅-alkoxy)-2-butanol or of 4-oxo-4-(hydroxy-C₃-C₅-alkoxy)-2-butanol, can be broken down physiologically, especially in the stomach and/or bowl, and release or generate the target molecule “3-hydroxybutyric acid” or its salts or esters as an active ingredient or active component.

In addition, the aforementioned carboxylic acid esters of 3-hydroxybutanoate, especially carboxylic acid esters of 4-oxo-4-(C₁-C₅-alkoxy)-2-butanol or of 4-oxo-4-(hydroxy-C₃-C₅-alkoxy)-2-butanol, also comprise an acceptable taste to ensure compatibility even when administered orally in larger quantities over a longer period of time (e. g. administration of 50 g daily dose or more).

Similarly, the production method according to the invention makes it possible to provide the carboxylic acid esters of 3-hydroxybutanoate, especially carboxylic acid esters of 4-oxo-4-(C₁-C₅-alkoxy)-2-butanol or of 4-oxo-4-(hydroxy-C₃-C₅-alkoxy)-2-butanol, free from toxic impurities.

In addition, with appropriate starting materials, the method can also be carried out enantioselectively. For example, according to the invention, the production method allows the biologically relevant form, i.e. the (R)-enantiomer, to be enriched, for example by enzyme catalysis or the specific selection of starting compounds (reactants), as not to burden the renal system of patients when administered orally (i.e. elimination via the kidneys). In principle, however, it is also possible, and under certain conditions may be useful, to enrich the(S)-enantiomer.

In addition, the production method according to the invention, including optional further processing or purification steps, can be operated economically and can also be implemented on a large scale.

Especially, the inventive production method uses commercially available starting compounds or starting compounds, which can be synthesized by simple processes that can be carried out on a large scale, and furthermore allows a relatively simple process management even in case of large-scale implementation.

In contrast to conventional prior art production methods, the production method according to the invention does not use complex starting materials or the use of protective groups and uses only a single step. Nevertheless, excellent yields are achieved in accordance with the invention, wherein the formation of by-products is minimized or avoided.

In addition, the inventive method is simple and economical. Especially, the method according to the invention is usually carried out in the absence of solvents and/or without any solvent (i.e. as a reaction in mass or as a reaction in substance or as a so-called bulk reaction); consequently, the reaction products obtained are not contaminated with solvent and no solvent has to be removed and disposed of or recycled in a costly and energy-intensive manner after the method or reaction has been carried out. Furthermore, no toxic by-products are formed.

The production method according to the invention usually results in a mixture of different carboxylic acid esters of 3-hydroxybutanoate, especially carboxylic acid esters of 4-oxo-4-(C₁-C₅-alkoxy)-2-butanol or of 4-oxo-4-(hydroxy-C₃-C₅-alkoxy)-2-butanol, i.e. in a mixture of at least two carboxylic acid esters of 3-hydroxybutanoate, especially carboxylic acid esters of 4-oxo-4-(C₁-C₅-alkoxy)-2-butanol or of 4-oxo-4-(hydroxy-C₃-C₅-alkoxy)-2-butanol, different from one another. The resulting raw reaction product or raw mixture can be purified by known methods, especially by removing any remaining starting compounds and/or any by-products present, and furthermore—if desired—can be separated by known methods, especially by distillation and/or chromatography (e.g. fractionation into the individual carboxylic acid esters of 3-hydroxybutanoate, i.e. separation of the corresponding monoester, diester, etc., or else fractionation into fractions with enriched and depleted portions of individuals etc.).

### BRIEF DESCRIPTION OF THE DRAWINGS

FIG. 1 provides a plot of the conversion-time-course of the reaction of succinic acid anhydride with ethyl 3-hydroxybutanoate.

FIG. 2 provides a plot of the conversion-time-course of the reaction of citric acid with ethyl 3-hydroxybutanoate.

FIG. 3 provides a plot of the conversion-time-course of the reaction of malic acid with ethyl 3-hydroxybutanoate.

### DETAILED DESCRIPTION OF THE INVENTION

As previously stated, according to the first aspect, the present invention relates to a method for producing carboxylic acid esters of 3-hydroxybutanoate, especially carboxylic acid esters of 4-oxo-4-(C₁-C₅-alkoxy)-2-butanol or of 4-oxo-4-(hydroxy-C₃-C₅-alkoxy)-2-butanol,
wherein at least one 3-hydroxybutanoate, especially a 4-oxo-4-(C₁-C₅-alkoxy)-2-butanol or a 4-oxo-4-(hydroxy-C₃-C₅-alkoxy)-2-butanol, of the general formula (I)

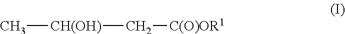

wherein, in general formula (I), the radical R¹ represents C₁-C₆-alkyl or hydroxy-C₃-C₅-alkyl, especially ethyl, butyl, pentyl, hydroxybutyl or hydroxypentyl, preferably ethyl, hydroxybutyl or hydroxypentyl, more preferably ethyl,
is reacted with at least one carboxylic acid (II), especially with at least one carboxylic acid comprising one or more carboxyl groups, preferably with at least one carboxylic acid comprising two or more carboxyl groups, especially in an esterification reaction and/or under esterification conditions,
so that, as a reaction product (III), one or more carboxylic acid esters of 3-hydroxybutanoate, especially one or more carboxylic acid esters of 4-oxo-4-(C₁-C₅-alkoxy)-2-butanol or of 4-oxo-4-(hydroxy-C₃-C₅-alkoxy)-2-butanol, are obtained.

According to a particular embodiment of the present invention, the compound of the general formula (I) can be used in racemic form or in the form of the (R)-enantiomer. The (R)-configuration refers to the chiral carbon atom in the 3-position of the compound of the general formula (I).

According to the invention, it is preferred when, in the general formula (I), the radical R¹ represents ethyl.

In other words, according to the invention, it is preferred that, as a compound of the general formula (I), 3-hydroxybutyrate (synonymously also referred to as 3-hydroxybutyric acid ethyl ester or 4-ethoxy-4-oxo-2-butanol) of the formula CH₃—CH(OH)—CH₂—C(O)OC₂H₅ is used.

This enables particularly efficient process control and high yields with minimized or suppressed by-product formation. In addition, the 3-hydroxybutyric acid ethyl ester or 4-ethoxy-4-oxo-2-butanol is also commercially available in large quantities and can especially be obtained on a large scale as a starting compound, e. g. by Claisen condensation of ethyl acetate.

According to a particular embodiment of the present invention, the carboxylic acid (II) may be used in the form of the free carboxylic acid, in the form of a salt of the carboxylic acid, in the form of an ester of the carboxylic acid or in the form of the carboxylic acid anhydride, especially in the form of the free carboxylic acid or in the form of the carboxylic acid anhydride.

According to another particular embodiment of the present invention, the carboxylic acid (II) may correspond to the general formula (IIa)

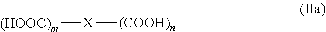

wherein, in the general formula (IIa),
   X represents an organic radical, especially a saturated or unsaturated organic radical comprising 1 to 10, preferentially 2 to 6 carbon atoms and optionally comprising 1 to 6 oxygen atoms; and
   the variable m represents an integer from 1 to 3; and
   the variable n represents an integer 0 or 1;
especially wherein at least one carboxyl group (COOH-group), preferentially two carboxyl groups (COOH-groups), is/are terminal and/or is/are a primary carboxyl group (COOH-group).

In this context, the radical X may also contain additional carboxyl groups (—COOH), so that the carboxylic acid (II) may contain up to 7 carboxyl groups in total.

In this context, it is particularly preferred if, in the general formula (IIa),
X represents a saturated or unsaturated and optionally mono- or polysubstituted, especially substituted with one or more hydroxyl radicals and/or carboxyl radical, organic radical comprising 2 to 6 carbon atoms; and
the variable m represents an integer 1; and
the variable n represents an integer 0 or 1;
   especially wherein at least one carboxyl group (COOH-group), preferentially two or both carboxyl groups (COOH-groups), is/are terminal and/or is/are a primary carboxyl group (COOOH-group).

Especially, by using a previously defined carboxylic acid with at least one, preferentially two, terminal or primary carboxyl groups, the esterification reaction between the carboxylic acid (II) and the 3-hydroxybutanoate can proceed particularly effectively and with minimized by-product formation without the need for extreme reaction conditions (e.g. very high temperature, very high pressure, etc.). In addition, the active ingredient density (i.e. especially 3-hydroxybutanoate density) can be influenced by the number of carboxyl groups present.

In the method according to the invention it is preferred if the carboxylic acid (II) is selected from the group of succinic acid, tartaric acid, lactic acid, citric acid, malic acid, adipic acid, fumaric acid and maleic acid and anhydrides thereof as well as combinations or mixtures thereof, especially is selected from the group of succinic acid, tartaric acid, lactic acid, citric acid, malic acid, adipic acid and fumaric acid and anhydrides thereof as well as combinations or mixtures thereof.

The previously mentioned carboxylic acids are particularly suitable for reaction with 3-hydroxybutanoates and are also commercially available.

Especially, it is preferred in the method according to the invention if the carboxylic acid (II) is a naturally occurring carboxylic acid or its anhydride or derivative, especially a reaction product, especially a carboxylic acid or its anhydride or derivative, especially a reaction product, occurring in human and/or animal metabolism.

Especially, it is advantageous in this context if carboxylic acids or anhydrides or derivatives thereof are used which occur in the citrate cycle, result from the citrate cycle or are associated with the citrate cycle. In this context, derivatives may, for example, represent salts which are obtainable by oxidation of a metabolic product (for example, from the citrate cycle). By using carboxylic acids or anhydrides or derivatives thereof, which are part of the human and/or animal metabolism or are reactant or product or intermediate of a human and/or animal metabolism, a further energy source (in addition to the keto body 3-hydroxybutyric acid or 3-hydroxybutanoate) can be provided to the human and/or animal body when using the reaction product according to the invention, and the reaction products are particularly suitable for use in or as medicaments, drugs or food and food products.

Furthermore, it may also be preferred in the context of the method according to the invention, if the carboxylic acid (II) is an ingredient, especially an additive, approved under food law.

Ingredients or additives approved under food law are those that are permitted for use in food in certain quantities and do not pose any health risks. A list of food additives is maintained throughout the EU, wherein each food additive is given its own label (so-called E-number). For example, the following carboxylic acids are included in the food additive list: succinic acid (E363), tartaric acid (E334), lactic acid (E270), citric acid (E330), malic acid (E296), adipic acid (E355) and fumaric acid (E297). These acids are all part of the citrate cycle or obtainable by oxidation of a metabolic product of the citrate cycle. The citrate cycle is a cycle of biochemical reactions that plays an important role in the metabolism of aerobic cells of living organisms and is mainly used for the oxidative degradation of organic substances for the purpose of energy production and the provision of intermediates for biosynthesis. Thus, the acids formed by degradation when using the reaction product (III) obtainable from the method according to the invention can be utilized by the body as another alternative source of energy.

According to a particular embodiment, the present invention refers, in accordance with this aspect of the invention, to a method for producing carboxylic acid esters of 3-hydroxybutanoate, especially carboxylic acid esters of 4-oxo-4-(C₁-C₅-alkoxy)-2-butanol or of 4-oxo-4-(hydroxy-C₃-C₅-alkoxy)-2-butanol, especially a method as described hereinabove,
wherein at least one 3-hydroxybutanoate, especially a 4-oxo-4-(C₁-C₅-alkoxy)-2-butanol or a 4-oxo-4-(hydroxy-C₃-C₅-alkoxy)-2-butanol, of the general formula (I)

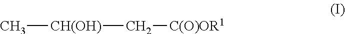

wherein, in the general formula (I), the radical R¹ represents C₁-C₅-alkyl or hydroxy-C₃-C₅-alkyl, especially ethyl, butyl, pentyl, hydroxybutyl or hydroxypentyl, preferably ethyl, hydroxybutyl or hydroxypentyl, more preferably ethyl,
is reacted with at least one carboxylic acid (II) selected from the group of succinic acid, tartaric acid, lactic acid, citric acid, malic acid, adipic acid, fumaric acid and maleic acid and anhydrides thereof as well as combinations or mixtures thereof, especially selected from the group of succinic acid tartaric acid, lactic acid, citric acid, malic acid, adipic acid and fumaric acid and anhydrides thereof as well as combinations or mixtures thereof, especially in an esterification reaction and/or under esterification conditions,
so that, as a reaction product (III), one or more carboxylic acid esters of 3-hydroxybutanoate, especially one or more carboxylic acid esters of 4-oxo-4-(C₁-C₅-alkoxy)-2-butanol or of 4-oxo-4-(hydroxy-C₃-C₅-alkoxy)-2-butanol, are obtained.

According to a further preferred embodiment, the present invention according to this aspect of the invention also refers to a method for producing carboxylic acid esters of 3-hydroxybutanoate, especially carboxylic acid esters of 4-oxo-4-(C₁-C₅-alkoxy)-2-butanol or of 4-oxo-4-(hydroxy-C₃-C₅-alkoxy)-2-butanol, especially a method as described hereinabove,
wherein at least one 3-hydroxybutanoate, especially a 4-oxo-4-(C₁-C₅-alkoxy)-2-butanol or a 4-oxo-4-(hydroxy-C₃-C₅-alkoxy)-2-butanol, of the general formula (I)

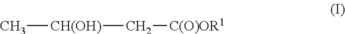

wherein, in the general formula (I), the radical R¹ represents ethyl,
is reacted with at least one carboxylic acid (II) selected from the group of succinic acid, tartaric acid, lactic acid, citric acid, malic acid, adipic acid, fumaric acid and maleic acid and anhydrides thereof as well as combinations or mixtures thereof, especially selected from the group of succinic acid tartaric acid, lactic acid, citric acid, malic acid, adipic acid and fumaric acid and anhydrides thereof as well as combinations or mixtures thereof, especially in an esterification reaction and/or under esterification conditions,
so that, as a reaction product (III), one or more carboxylic acid esters of 3-hydroxybutanoate, especially one or more carboxylic acid esters of 4-oxo-4-(C₁-C₅-alkoxy)-2-butanol or of 4-oxo-4-(hydroxy-C₃-C₅-alkoxy)-2-butanol, are obtained.

According to a particular embodiment of the present invention, the reaction may be carried out in the absence of solvents and/or without any solvent. This means that the reaction is carried out as a reaction in mass or as a reaction in substance or as a so-called bulk reaction. This has the advantage that the reaction products obtained are not contaminated with solvent and that no solvent has to be removed and disposed of or recycled in a costly and energy-intensive manner after the method or reaction has been carried out.

Surprisingly, the method or reaction nevertheless proceeds with high conversion and yields and at least essentially without significant by-product formation.

According to another particular embodiment of the present invention, the reaction may be carried out in the absence of a catalyst and/or without any catalyst, or alternatively the reaction may be carried out in the presence of a catalyst, especially an enzyme and/or a metal-containing and/or metal-based, acidic or basic catalyst. Especially, the catalyst may be recycled after the reaction.

In the method according to the invention, it is particularly preferred if the reaction is carried out in the absence of solvents and/or without any solvent and if the reaction is carried out in the absence of a catalyst and/or without any catalyst.

Due to the absence of solvents and the absence of a catalyst, the reaction products cannot be contaminated by incompletely removed solvent or incompletely removed catalyst. In addition, the energy-intensive and costly removal or separation steps are eliminated. Surprisingly, the method according to the invention in accordance with this preferred embodiment is nevertheless economical and results in high conversions without significant by-product formation.

As previously stated, according to a particular embodiment of the method of the invention, the reaction may be carried out in the absence of a catalyst and/or without any catalyst.

Provided that the reaction is carried out in the absence of a catalyst and/or without any catalyst, it is preferred if the reaction is carried out in the absence of a catalyst and/or without any catalyst at temperatures in the range of from 20° C. to 160° C., especially in the range of from 50° C. to 150° C., preferentially in the range of from 70° C. to 140° C., more preferably in the range of from 80° C. to 135° C., even more preferably in the range of from 100° C. to 130° C.

In the case of conversion in the absence of a catalyst, the applied pressure range may also vary within wide ranges. Especially, the reaction can be carried out in the absence of a catalyst and/or without any catalyst at a pressure in the range of from 0.0001 bar to 10 bar, especially in the range of from 0.001 bar to 5 bar, preferentially in the range of from 0.01 bar to 2 bar, more preferably in the range of from 0.05 bar to 1 bar, even more preferably at about 1 bar.

When reacting in the absence of a catalyst, it is preferred if the reaction is carried out in the presence of an inert gas, especially in the presence of helium, argon or nitrogen, preferably in the presence of nitrogen. Especially, undesirable side reactions, especially due to oxidation or hydrolysis, may be thus prevented.

Alternatively to this particular embodiment, however, it is also possible to carry out the reaction, for example, in the presence of an enzyme as a catalyst.

In this context, the enzyme can especially be selected from synthetases (ligases), catalases, esterases, lipases and combinations thereof. According to the invention, synthetases (synonymously ligases) are especially enzymes from the class of ligases; ligases are enzymes which catalyze the linking of two or more molecules by a covalent bond. Catalases in the sense of the present invention are especially enzymes which are capable of converting hydrogen peroxide to oxygen and water. The term esterases refers in particular to enzymes which are capable of hydrolytically splitting esters into alcohol and acid (saponification); these are thus especially hydrolases, wherein fat splitting esterases are also called lipases. Lipases in the sense of the present invention are especially enzymes which are capable of splitting free fatty acids from lipids such as glycerides (lipolysis).

In this context, the enzyme used as catalyst can especially be derived from Candida antarctica, Mucor miehei (Rhizomucor miehei), Thermomyces lanuginosus, Candida rugosa, Aspergillus oryzae, Pseudomonas cepacia, Pseudomonas fluorescens, Rhizopus delemar and Pseudomonas sp. and combinations thereof, preferentially from Candida antarctica, Mucor miehei (Rhizomucor miehei) and Thermomyces lanuginosus.

According to a specific embodiment, the enzyme can be used in immobilized form, especially immobilized on a carrier, preferentially on a polymeric carrier, preferably on a polymeric organic carrier, more preferably with hydrophobic properties, even more preferably on a poly(meth)acrylic resin-based carrier.

In the context of the present invention, it is preferred that in the case that an enzyme is used as a catalyst, the enzyme is recycled after the reaction.

If the reaction is carried out in the presence of an enzyme as a catalyst within the framework of the inventive production method, it is preferred if the reaction is carried out at temperatures in the range of from 10° C. to 80° C., especially in the range of from 20° C. to 80° C., preferentially in the range of from 25° C. to 75° C., more preferably in the range of from 45° C. to 75° C., even more preferably in the range of from 50° C. to 70° C.

In case of using an enzyme as a catalyst, the amount of the enzyme used can vary within wide ranges. Especially, the enzyme can be used in amounts, based on the total amount of the starting compounds (I) and (II), in the range of from 0.001% by weight to 20% by weight, especially in the range of from 0.01% by weight to 15% by weight, preferentially in the range of from 0.1% by weight to 15% by weight, preferably in the range of from 0.5% by weight to 10% by weight. Nevertheless, it may be necessary to deviate from the above-mentioned amounts in individual cases or for specific applications without leaving the scope of the present invention.

If, according to a particular embodiment of the present invention, the reaction is carried out in the presence of an enzyme as a catalyst, the applied pressure range may also vary within wide ranges. Typically, the reaction in the presence of an enzyme can be carried out at a pressure in the range of from 0.0001 bar to 10 bar, especially in the range of from 0.001 bar to 5 bar, preferentially in the range of from 0.01 bar to 2 bar, more preferably in the range of from 0.05 bar to 1 bar, even more preferably at about 0.5 bar.

According to the particular embodiment of the present invention, according to which the reaction is carried out in the presence of an enzyme as catalyst, it is preferred if the reaction is carried out in the presence of an enzyme in the presence of an inert gas, especially in the presence of helium, argon or nitrogen, preferably in the presence of nitrogen. As previously stated in connection with the reaction in the absence of a catalyst, undesirable side reactions, especially due to oxidation or hydrolysis, may be prevented by the reaction in the presence of an inert gas.

According to another alternative embodiment of the present invention, the reaction can be carried out, for example, in the presence of a metal-containing and/or metal-based, acidic or basic catalyst.

According to this alternative embodiment of the present invention, according to which the reaction is carried out in the presence of a metal-containing and/or metal-based, acidic or basic catalyst, the catalyst can especially be selected from (i) basic catalysts, especially alkali or alkaline earth hydroxides and alkali or alkaline earth alcoholates, such as NaOH, KOH, LiOH, Ca(OH)₂, NaOMe, KOMe and Na(OBu-tert.), (ii) acidic catalysts, especially mineral acids, and organic acids, such as sulfuric acid, hydrochloric acid, phosphoric acid, nitric acid, sulfonic acids, methane sulfonic acid, para-toluene sulfonic acid and carboxylic acids, (iii) Lewis acids, especially Lewis acids based on titanium, tin, zinc and aluminum compounds, such as titanium tetrabutylate, tin acids, zinc acetate, aluminum trichloride and aluminum tri-isopropyl, and (iv) heterogeneous catalysts, especially based on mineral silicates, germanates, carbonates and aluminum oxides, such as zeolites, montmorillonites, mordenites, hydrotalcites and aluminas, and combinations thereof.

In this embodiment, a Lewis acid based on titanium, tin, zinc and aluminum compounds, such as titanium tetrabutylate, tin acids, zinc acetate, aluminum trichloride and aluminum tri-isopropyl, may be used as a catalyst.

Especially, also according to this embodiment it is preferred if the metal-containing and/or metal-based, acidic or basic catalyst is recycled after the reaction.

Also according to the particular embodiment of the present invention, according to which the reaction is carried out in the presence of a metal-containing and/or metal-based, acidic or basic catalyst, the temperatures can be varied within wide ranges. Especially, the reaction can be carried out in the presence of a metal-containing and/or metal-based, acidic or basic catalyst at temperatures in the range of from 20° C. to 160° C., especially in the range of from 50° C. to 150° C., preferentially in the range of from 70° C. to 140° C., more preferably in the range of from 80° C. to 135° C., even more preferably in the range of from 100° C. to 130° C.

Furthermore, also according to this embodiment, the catalyst (i.e. the metal-containing and/or metal-based, acidic or basic catalyst) can also be varied within wide quantity ranges: For example, the catalyst can be used in amounts, based on the total amount of the starting compounds (I) and (II), in the range of from 0.01 to 30% by weight, especially in the range of from 0.05 to 15% by weight, preferentially in the range of from 0.1 to 15% by weight, preferably in the range of from 0.2 to 10% by weight. Nevertheless, it is possible to deviate from the above-mentioned amounts for specific applications or individual cases without leaving the scope of the present invention.

Moreover, according to this particular embodiment of the present invention, according to which the reaction is carried out in the presence of a metal-containing and/or metal-based, acidic or basic catalyst, the pressure range can equally vary within a wide range: Especially, the reaction can be carried out in the presence of a metal-containing and/or metal-based, acidic or basic catalyst at a pressure in the range of from 0.0001 bar to 10 bar, especially in the range of from 0.001 bar to 5 bar, preferentially in the range of from 0.01 bar to 2 bar, more preferably in the range of from 0.05 bar to 1 bar, even more preferably at about 1 bar.

Furthermore, also according to this particular embodiment of the present invention, according to which the reaction is carried out in the presence of a metal-containing and/or metal-based, acidic or basic catalyst, it is preferred if the reaction is carried out in the presence of an inert gas, especially in the presence of helium, argon or nitrogen, preferably in the presence of nitrogen. As previously stated, the reaction in the presence of an inert gas prevents undesirable side reactions, especially side reactions due to oxidation or hydrolysis.

As far as the total quantity of starting materials or starting compounds is concerned, this can also be varied within wide ranges.

Taking into account process economy and optimization of the course of the method, especially with regard to the minimization of by-products, it is advantageous if the 3-hydroxybutanoat of the general formula (I), based on the carboxyl groups of the carboxylic acid (II) is used in molar amounts in a range of from equimolar amount up to a molar excess of 200 mol-%, especially in a range of from equimolar amount up to a molar excess of 150 mol-%, preferentially in a range of from equimolar amount up to a molar excess of 100 mol-%.

Similarly, taking into account process economy and optimization of the course of the method, especially with regard to minimizing by-products, it is advantageous if the 3-hydroxybutanoate of the general formula (I) and the carboxylic acid (II) are used in a molar ratio of 3-hydroxybutanoate of the general formula (I)/carboxylic acid (II) in a range of from 1:1 to 10:1, especially in a range of from 2:1 to 8:1, preferentially in a range of from 3:1 to 6:1.

Typically, in the method according to the invention, during the reaction of 3-hydroxybutanoate of the general formula (I) with a carboxylic acid (II) in the form of the free acid, water is formed simultaneously. Especially, it is preferred if the water is withdrawn from the reaction, especially continuously withdrawn, especially by means of preferentially continuous, especially distillative or adsorptive removal.

Usually, in the method according to the invention, during the reaction of 3-hydroxybutanoate of the general formula (I) with a carboxylic acid (II) in the form of the anhydride, one mol of the corresponding free carboxylic acid (II) is formed per mol of the anhydride used. Especially, the resulting free carboxylic acid (II) is reacted with 3-hydroxybutanoate of the general formula (I) or, after the reaction has taken place, is removed and optionally recycled, especially depending on the amounts and/or ratios of the starting compounds (I) and (II) used.

However, when using internal or cyclic anhydrides (such as succinic anhydride or maleic anhydride), the ring is opened and no cleavage product is formed, so that the reaction product comprises a terminal free acid. This terminal free acid can then optionally react again with a 3-hydroxybutanoate of the general formula (I). This method is illustrated below using the example of the reaction of maleic anhydride with 3-hydroxybutyric acid ethyl ester:

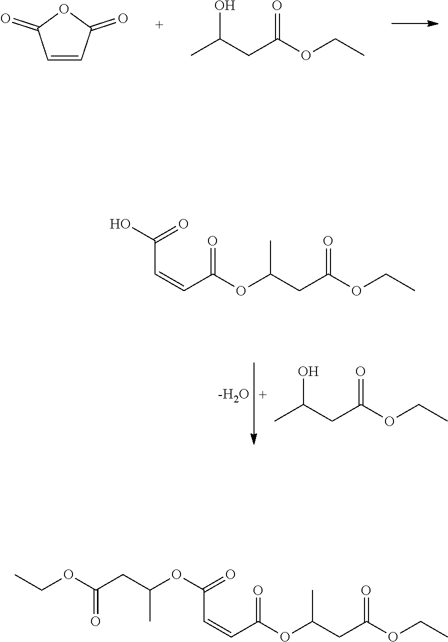

In the method according to the invention, the composition of the reaction product, especially the presence of the various carboxylic acid esters of 3-hydroxybutanoate (III), and their proportion in the case of a mixture, may be controlled and/or regulated by means of the reaction conditions, especially by selecting the reaction temperature (conversion temperature) and/or selecting the reaction pressure (conversion pressure) and/or absence of or providing a catalyst and selecting such catalyst with respect to the type and/or amount and/or selecting the amounts of the starting compounds (reactants) and/or providing the removal of the by-products, especially water, that may be formed.

Thus, it is possible to tailor the composition of the product or product mixture depending on the application. Especially, for example, the number of substituted 3-hydroxybutyric acid radicals (3-hydroxybutanoates) can be adjusted so that the density of keto bodies in the form of 3-hydroxybutanoates per molecule can be adjusted in a targeted manner.

After the reaction, the reaction product obtained can be subjected to further purification or work-up steps.

In this context, the reaction product obtained can be fractionated after the reaction has been performed, especially fractionated by distillation.

Also, unreacted starting compounds (1) and/or (II) can be separated from the reaction product and subsequently recycled.

According to a special embodiment of the production method according to the invention, it is especially possible to proceed in such a way that hydroxyl groups and/or carboxyl groups still present in the reaction product after the reaction has been performed are at least partially, preferentially completely, functionalized, especially esterified.

In other words, the reaction can be followed by a partial, especially complete functionalization, especially esterification, of hydroxyl groups and/or carboxyl groups still present.

In this particular embodiment of the method according to the invention, the functionalization, especially the esterification of hydroxyl groups and/or carboxyl groups still present, can be carried out by reaction with a carboxylic acid anhydride of, for example, C₂-C₃₀-carboxylic acids or C₂-C₃₀-fatty acids in the case of free hydroxyl groups or C₂-C₃₀-fatty alcohols in the case of free carboxyl groups. These may be linear or branched, saturated or mono- or polyunsaturated C₂-C₃₀-carboxylic anhydrides or C₂-C₃₀-fatty acids or C₂-C₃₀-fatty alcohols. In this context, hydroxyl groups still present can especially be reacted with carboxylic acid anhydrides or fatty acids, and carboxyl groups still present can especially be reacted with fatty alcohols.

Within the scope of the inventive method, as a reaction product, one or more carboxylic acid esters of 3-hydroxybutanoate, especially one or more carboxylic acid esters of 4-oxo-4-(C₁-C₅-alkoxy)-2-butanol or of 4-oxo-4-(hydroxy-C₃-C₅-alkoxy)-2-butanol, of the general formula (IIIa)

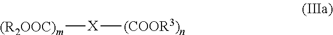

may be obtained and/or may be obtainable, wherein, in the general formula (IIIa),
   X represents an organic radical, especially a saturated or unsaturated organic radical comprising 1 to 10, preferentially 2 to 6 carbon atoms and optionally comprising 1 to 6 oxygen atoms;
   the variable m represents an integer from 1 to 3;
   the variable n represents an integer 0 or 1; and
   R² and R³, each independently of one another, represent hydrogen or a radical —CH(CH₃)—CH₂—C(O)OR¹, wherein the radical R¹ represents C₁-C₅-alkyl or hydroxy-C₃-C₅-alkyl, especially ethyl, butyl, pentyl, hydroxybutyl or hydroxypentyl, preferably ethyl, hydroxybutyl or hydroxypentyl, more preferably ethyl, however, with the proviso that at least one, preferentially at least two, of the radicals R² and R³ represent a radical —CH(CH₃)—CH₂—C(O)OR¹.

Especially, at least one of the radicals R²OOC— and/or R³OOC—, preferentially two of the radicals R²OOC— and R³OOC—, may be terminal and/or may be a primary radical.

As already delineated hereinabove, in this context, the radical X may also comprise further carboxyl groups or carboxyl groups substituted with —CH(CH₃)—CH₂—C(O)OR¹ (i.e. radicals —COO—CH(CH₃)—CH₂—C(O)OR¹).

Especially, according to the inventive production method, as a reaction product (III), one or more carboxylic acid esters of 3-hydroxybutanoate, especially one or more carboxylic acid esters of 4-oxo-4-(C₁-C₅-alkoxy)-2-butanol or of 4-oxo-4-(hydroxy-C₃-C₅-alkoxy)-2-butanol, of the general formula (IIIa)

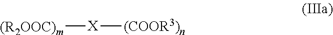

may be obtained may be obtainable, wherein, in the general formula (IIIa),
   X represents a saturated or unsaturated and optionally mono- or polysubstituted, especially substituted by one or more hydroxyl radicals and/or radicals —O—C(O)—CH₂—CH(OH)—CH₃ and/or carboxyl radicals and/or radicals —C(O)—O—CH(CH₃)—CH₂—C(O)OR¹, organic radical comprising 2 to 6 carbon atoms;
   the variable m represents an integer 1;
   the variable n represents an integer 0 or 1; and
   R² and R³, each independently of one another, represent hydrogen or a radical —CH(CH₃)—CH₂—C(O)OR¹, wherein the radical R¹ is C₁-C₅-alkyl or hydroxy-C₃-C₅-alkyl, especially ethyl, butyl, pentyl, hydroxybutyl or hydroxypentyl, preferably ethyl, hydroxybutyl or hydroxypentyl, more preferably ethyl, however, with the proviso that at least one, preferentially both of the radicals R² and R³ is/are a radical —CH(CH₃)—CH₂—C(O)OR¹.

Especially, at least one of the radicals R²OOC— and/or R³OOC—, preferentially two or both of the radicals R²OOC— and R³OOC—, may be terminal and/or may be a primary radical.

As previously stated, in this context the radical X may also contain further carboxyl groups or carboxyl groups substituted with —CH(CH₃)—CH₂—C(O)OR¹ (i.e. radicals —COO—CH(CH₃)—CH₂—C(O)OR¹).

According to a particular embodiment of the method according to the invention, as a reaction product (III), one or more carboxylic acid esters of 3-hydroxybutanoate, especially one or more carboxylic acid esters of 4-oxo-4-(C₁-C₅-alkoxy)-2-butanol or of 4-oxo-4-(hydroxy-C₃-C₅-alkoxy)-2-butanol, of the general formula (IIIb)

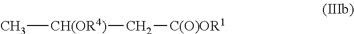

may be obtained and/or may be obtainable, wherein, in the general formula (IIIb),
   the radical R¹ represents C₁-C₅-alkyl or hydroxy-C₃-C₅-alkyl, especially ethyl, butyl, pentyl, hydroxybutyl or hydroxypentyl, preferably ethyl, hydroxybutyl or hydroxypentyl, more preferably ethyl, and
   the radical R⁴ is derived from a carboxylic acid selected from the group of succinic acid, tartaric acid, lactic acid, citric acid, malic acid, adipic acid, fumaric acid and maleic acid as well as combinations or mixtures thereof, especially selected from the group of succinic acid, tartaric acid, lactic acid, citric acid, malic acid, adipic acid and fumaric acid as well as combinations or mixtures thereof.

Especially, in the case of succinic acid, tartaric acid, citric acid, malic acid, adipic acid, fumaric acid and maleic acid, further carboxyl groups present may be completely or partially esterified with a radical —CH(CH₃)—CH₂—C(O)OR¹ with R¹ as defined hereinabove.

In this context, “derived from” means that the radical R⁴ is formed from the carboxylic acids cited; especially, the hydrogen of the carboxyl is esterified by esterification; i.e. in each case the carboxylate radical of the corresponding acid is present as the radical R⁴ (i.e. the radical R⁴ is a succinate radical in the case of succinic acid, a tartrate radical in the case of tartaric acid, a lactate radical in the case of lactic acid, a citrate radical in the case of citric acid, a malate radical in the case of malic acid, an adipate radical in the case of adipic acid, a fumarate radical in the case of fumaric acid and a maleate radical in the case of maleic acid).

According to a further particular embodiment of the method according to the invention, as a reaction product (III), one or more carboxylic acid esters of 3-hydroxybutanoate, especially one or more carboxylic acid esters of 4-oxo-4-(C₁-C₅-alkoxy)-2-butanol or of 4-oxo-4-(hydroxy-C₃-C₅-alkoxy)-2-butanol, of the general formula (IIIb)

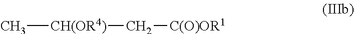

may be obtained and/or may be obtainable, wherein, in the general formula (IIIb),
   the radical R¹ represents C₁-C₅-alkyl or hydroxy-C₃-C₅-alkyl, especially ethyl, butyl, pentyl, hydroxybutyl or hydroxypentyl, preferably ethyl, hydroxybutyl or hydroxypentyl, more preferably ethyl, and
   the radical R⁴ represents one or more of the following radicals

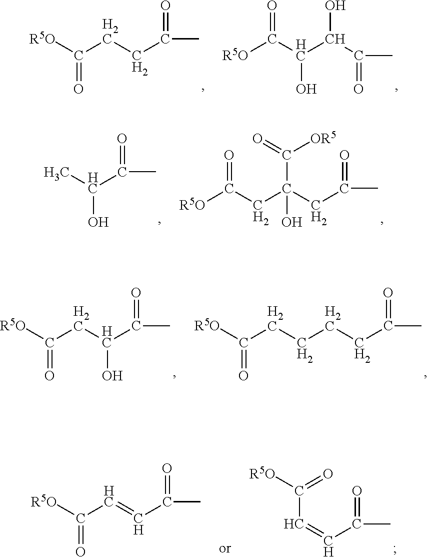

wherein, in the above radicals, the radical R⁵ represents hydrogen or a radical —CH(CH₃)—CH₂—C(O)OR¹ with R¹ as defined hereinabove.

According to yet another particular embodiment of the method according to the invention, as a reaction product, a mixture of at least two different carboxylic acid esters of 3-hydroxybutanoate (III), especially carboxylic acid esters of 4-oxo-4-(C₁-C₅-alkoxy)-2-butanol or of 4-oxo-4-(hydroxy-C₃-C₅-alkoxy)-2-butanol, especially as defined hereinabove, may be obtained.

A further object—according to a second aspect of the present invention—is a (chemical) product or product mixture, preferentially a carboxylic acid ester of 3-hydroxybutanoate, especially carboxylic acid ester of 4-oxo-4-(C₁-C₅-alkoxy)-2-butanol or of 4-oxo-4-(hydroxy-C₃-C₅-alkoxy)-2-butanol, or a mixture of several carboxylic acid esters of 3-hydroxybutanoate, especially a mixture of several carboxylic acid esters of 4-oxo-4-(C₁-C₅-alkoxy)-2-butanol or of 4-oxo-4-(hydroxy-C₃-C₅-alkoxy)-2-butanol, obtainable by the method described hereinabove.

According to a particular embodiment of this aspect, the object of the present invention is a reaction product, especially a (chemical) product or product mixture, especially a reaction product as previously defined,
wherein the reaction product (III) comprises one or more carboxylic acid esters of 3-hydroxybutanoate, especially one or more carboxylic acid esters of 4-oxo-4-(C₁-C₅-alkoxy)-2-butanol or of 4-oxo-4-(hydroxy-C₃-C₅-alkoxy)-2-butanol, of the general formula (IIIa)

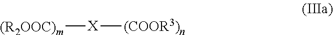

wherein, in the general formula (IIIa),
   X represents an organic radical, especially a saturated or unsaturated organic radical comprising 1 to 10, preferentially 2 to 6 carbon atoms and optionally comprising 1 to 6 oxygen atoms;
   the variable m represents an integer from 1 to 3;
   the variable n represents an integer 0 or 1; and
   R² and R³, each independently of one another, represent hydrogen or a radical —CH(CH₃)—CH₂—C(O)OR¹, wherein the radical R¹ represents C₁-C₅-alkyl or hydroxy-C₃-C₅-alkyl, especially ethyl, butyl, pentyl, hydroxybutyl or hydroxypentyl, preferably ethyl, hydroxybutyl or hydroxypentyl, more preferably ethyl, however, with the proviso that at least one, preferentially at least two, of the radicals R² and R³ represent a radical —CH(CH₃)—CH₂—C(O)OR¹;

especially wherein at least one of the radicals R²OOC— and/or R³OOC—, preferentially two of the radicals R²OOC— and R³OOC—, is/are terminal and/or a primary radical.

In this context, as previously stated, the radical X may also contain further carboxyl groups or carboxyl groups substituted with —CH(CH₃)—CH₂—C(O)OR¹ (i.e. radicals —COO—CH(CH₃)—CH₂—C(O)OR¹).

According to another particular embodiment of the present invention, the reaction product (III) may comprise one or more carboxylic acid esters of 3-hydroxybutanoate, especially one or more carboxylic acid esters of 4-oxo-4-(C₁-C₅-alkoxy)-2-butanol or of 4-oxo-4-(hydroxy-C₃-C₅-alkoxy)-2-butanol, of the general formula (IIIa)

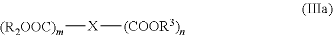

wherein, in the general formula (IIIa),
   X represents a saturated or unsaturated and optionally mono- or polysubstituted, especially substituted with one or more hydroxyl radicals and/or radicals —O—C(O)—CH₂—CH(OH)—CH₃ and/or carboxyl radicals and/or radicals —C(O)—O—CH(CH₃)—CH₂—C(O)OR¹, organic radical comprising 2 to 6 carbon atoms;
   the variable m represents an integer 1;
   the variable n represents an integer 0 or 1; and
   R² and R³, each independently of one another, represent hydrogen or a radical —CH(CH₃)—CH₂—C(O)OR¹, wherein the radical R¹ represents C₁-C₅-alkyl or hydroxy-C₃-C₅-alkyl, especially ethyl, butyl, pentyl, hydroxybutyl or hydroxypentyl, preferably ethyl, hydroxybutyl or hydroxypentyl, more preferably ethyl, however, with the proviso that at least one, preferentially both, of the radicals R² and R³ is/are a radical —CH(CH₃)—CH₂—C(O)OR¹.

Especially, at least one of the radicals R²OOC— and/or R³OOC—, preferentially two or both of the radicals R²OOC— and R³OOC—, may be terminal and/or may be a primary radical.

In this context, the radical X may also contain further carboxyl groups or carboxyl groups substituted with —CH(CH₃)—CH₂—C(O)OR¹ (i.e. radicals —COO—CH(CH₃)—CH₂—C(O)OR¹).

According to yet another particular embodiment of the present invention, the reaction product (III) may comprise one or more carboxylic acid esters of 3-hydroxybutanoate, especially one or more carboxylic acid esters of 4-oxo-4-(C₁-C₅-alkoxy)-2-butanol or of 4-oxo-4-(hydroxy-C₃-C₅-alkoxy)-2-butanol, of the general formula (IIIb)

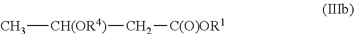

wherein, in the general formula (IIIb),
   the radical R¹ represents C₁-C₅-alkyl or hydroxy-C₃-C₅-alkyl, especially ethyl, butyl, pentyl, hydroxybutyl or hydroxypentyl, preferably ethyl, hydroxybutyl or hydroxypentyl, more preferably ethyl, and
the radical R⁴ is derived from a carboxylic acid selected from the group of succinic acid, tartaric acid, lactic acid, citric acid, malic acid, adipic acid, fumaric acid and maleic acid as well as combinations or mixtures thereof, especially selected from the group of succinic acid, tartaric acid, lactic acid, citric acid, malic acid, adipic acid and fumaric acid as well as combinations or mixtures thereof.

Especially, in the case of succinic acid, tartaric acid, citric acid, malic acid, adipic acid, fumaric acid and maleic acid, further carboxyl groups present may be completely or partially esterified with a radical —CH(CH₃)—CH₂—C(O)OR¹ with R¹ as defined hereinabove.

As previously stated, in this context “derived from” means that the radical R⁴ is formed from the carboxylic acids mentioned, especially the hydrogen of the carboxyl is esterified by esterification; i.e. in each case the carboxylate radical of the corresponding acid is present as the radical R⁴ (i.e. the radical R⁴ is a succinate radical in the case of succinic acid, a tartrate radical in the case of tartaric acid, a lactate radical in the case of lactic acid, a citrate radical in the case of citric acid, a malate radical in the case of malic acid, an adipate radical in the case of adipic acid, a fumarate radical in the case of fumaric acid and a maleate radical in the case of maleic acid).

According to another particular embodiment of the present invention, the reaction product (III) may comprise one or more carboxylic acid esters of 3-hydroxybutanoate, especially one or more carboxylic acid esters of 4-oxo-4-(C₁-C₅-alkoxy)-2-butanol or of 4-oxo-4-(hydroxy-C₃-C₅-alkoxy)-2-butanol, of the general formula (IIIb)

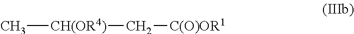

wherein, in the general formula (IIIb),
   the radical R¹ represents C₁-C₅-alkyl or hydroxy-C₃-C₅-alkyl, especially ethyl, butyl, pentyl, hydroxybutyl or hydroxypentyl, preferably ethyl, hydroxybutyl or hydroxypentyl, more preferably ethyl, and
   the radical R⁴ represents one or more of the following radicals

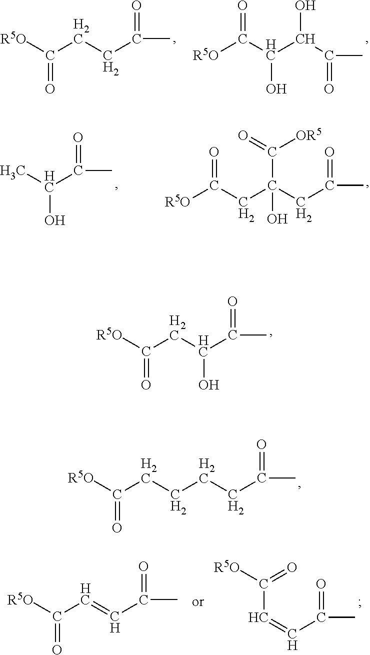

wherein, in the above radicals, R⁵ represents hydrogen or a radical —CH(CH₃)—CH₂—C(O)OR¹ with R¹ as defined hereinabove.

According to a particular embodiment, the reaction product may comprise a mixture of at least two different carboxylic acid esters of 3-hydroxybutanoate (III), especially carboxylic acid esters of 4-oxo-4-(C₁-C₅-alkoxy)-2-butanol or of 4-oxo-4-(hydroxy-C₃-C₅-alkoxy)-2-butanol, especially as defined hereinabove.

Thus, object of the present invention is also a carboxylic acid ester of 3-hydroxybutanoate (=carboxylic acid esters of 4-oxo-2-butanol), especially carboxylic acid ester of 4-oxo-4-(C₁-C₅-alkoxy)-2-butanol or of 4-oxo-4-(hydroxy-C₃-C₅-alkoxy)-2-butanol, of the general formula (IIIa)

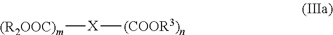

wherein, in the general formula (IIIa),
   X represents an organic radical, especially a saturated or unsaturated organic radical comprising 1 to 10, preferentially 2 to 6, carbon atoms and optionally comprising 1 to 6 oxygen atoms;
   the variable m represents an integer from 1 to 3;
   the variable n represents an integer 0 or 1; and
   R² and R³, each independently of one another, represent hydrogen or a radical —CH(CH₃)—CH₂—C(O)OR¹, wherein the radical R¹ represents C₁-C₅-alkyl or hydroxy-C₃-C₅-alkyl, especially ethyl, butyl, pentyl, hydroxybutyl or hydroxypentyl, preferably ethyl, hydroxybutyl or hydroxypentyl, more preferably ethyl, however, with the proviso that at least one, preferentially at least two, of the radicals R² and R³ represent a radical —CH(CH₃)—CH₂—C(O)OR¹.

Especially, at least one of the radicals R²OOC— and/or R³OOC—, preferentially two of the radicals R²OOC— and R³OOC—, may be terminal and/or may be a primary radical.

As previously stated, the radical X may also contain other carboxyl groups or carboxyl groups substituted with —CH(CH₃)—CH₂—C(O)OR¹ (i.e. radicals —COO—CH(CH₃)—CH₂—C(O)OR¹).

According to a particular embodiment, the carboxylic acid ester of 3-hydroxybutanoate, especially carboxylic acid ester of 4-oxo-4-(C₁-C₅-alkoxy)-2-butanol or of 4-oxo-4-(hydroxy-C₃-C₅-alkoxy)-2-butanol, may correspond to the general formula (IIIa)

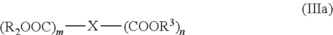

wherein, in the general formula (IIIa),
   X represents a saturated or unsaturated and optionally mono- or polysubstituted, especially substituted with one or more hydroxyl radicals and/or radicals —O—C(O)—CH₂—CH(OH)—CH₃ and/or carboxyl radicals and/or radicals —C(O)—O—CH(CH₃)—CH₂—C(O)OR¹, organic radical comprising 2 to 6 carbon atoms;
   the variable m represents an integer 1;
   the variable n represents an integer 0 or 1; and
   R² and R³, each independently of one another, represent hydrogen or a radical —CH(CH₃)—CH₂—C(O)OR¹, wherein the radical R¹ represents C₁-C₅-alkyl or hydroxy-C₃-C₅-alkyl, especially ethyl, butyl, pentyl, hydroxybutyl or hydroxypentyl, preferably ethyl, hydroxybutyl or hydroxypentyl, more preferably ethyl, however, with the proviso that at least one, preferentially both, of the radicals R² and R³ is/are a radical —CH(CH₃)—CH₂—C(O)OR¹.

Especially, at least one of the radicals R²OOC— and/or R³OOC—, preferentially two or both of the radicals R²OOC— and R³OOC—, may be terminal and/or may be a primary radical.

The radical X may also contain other carboxyl groups or carboxyl groups substituted with —CH(CH₃)—CH₂—C(O)OR¹ (i.e. radicals —COO—CH(CH₃)—CH₂—C(O)OR¹).

According to a further particular embodiment, the carboxylic acid ester of 3-hydroxybutanoate, especially carboxylic acid ester of 4-oxo-4-(C₁-C₅-alkoxy)-2-butanol or of 4-oxo-4-(hydroxy-C₃-C₅-alkoxy)-2-butanol, may correspond to the general formula (IIIb)

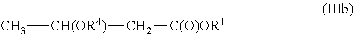

wherein, in the general formula (IIIb),
   the radical R¹ represents C₁-C₅-alkyl or hydroxy-C₃-C₅-alkyl, especially ethyl, butyl, pentyl, hydroxybutyl or hydroxypentyl, preferably ethyl, hydroxybutyl or hydroxypentyl, more preferably ethyl, and
   the radical R⁴ is derived from a carboxylic acid selected from the group of succinic acid, tartaric acid, lactic acid, citric acid, malic acid, adipic acid, fumaric acid and maleic acid as well as combinations or mixtures thereof, especially selected from the group of succinic acid, tartaric acid, lactic acid, citric acid, malic acid, adipic acid and fumaric acid as well as combinations or mixtures thereof;
especially wherein, in the case of succinic acid, tartaric acid, citric acid, malic acid, adipic acid, fumaric acid and maleic acid, further carboxyl groups present are esterified completely or partially with a radical —CH(CH₃)—CH₂—C(O)OR¹ with R¹ as defined hereinabove.

As previously stated, “derived from” means that the radical R⁴ is formed from the carboxylic acids mentioned, especially the hydrogen of the carboxyl is esterified by esterification; i.e. in each case the carboxylate radical of the corresponding acid is present as the radical R⁴ (i.e. the radical R⁴ is a succinate radical in the case of succinic acid, a tartrate radical in the case of tartaric acid, a lactate radical in the case of lactic acid, a citrate radical in the case of citric acid, a malate radical in the case of malic acid, an adipate radical in the case of adipic acid, a fumarate radical in the case of fumaric acid and a maleate radical in the case of maleic acid).

According to a preferred embodiment of the present invention, the carboxylic acid ester of 3-hydroxybutanoate, especially carboxylic acid ester of 4-oxo-4-(C₁-C₅-alkoxy)-2-butanol or of 4-oxo-4-(hydroxy-C₃-C₅-alkoxy)-2-butanol, may correspond to the general formula (IIIb)

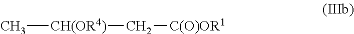

wherein, in the general formula (IIIb),
   the radical R¹ represents C₁-C₅-alkyl or hydroxy-C₃-C₅-alkyl, especially ethyl, butyl, pentyl, hydroxybutyl or hydroxypentyl, preferably ethyl, hydroxybutyl or hydroxypentyl, more preferably ethyl, and
   the radical R⁴ represents one or more of the following radicals

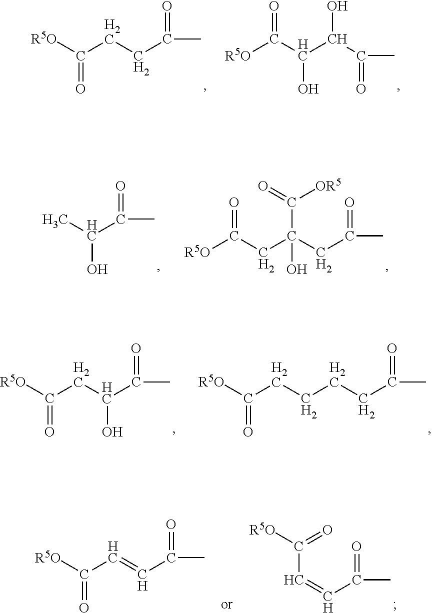

wherein, in the above radicals, the radical R⁵ represents hydrogen or a radical —CH(CH₃)—CH₂—C(O)OR¹ with R¹ as defined hereinabove.

Furthermore, according to a particular embodiment, another object of the present invention is a mixture comprising at least two different carboxylic acid esters of 3-hydroxybutanoate (III), especially carboxylic acid esters of 4-oxo-4-(C₁-C₅-alkoxy)-2-butanol or of 4-oxo-4-(hydroxy-C₃-C₅-alkoxy)-2-butanol, especially as defined hereinabove.

The reaction product obtainable according to the inventive method or the inventive reaction product as defined hereinabove, respectively, and/or the carboxylic acid ester of 3-hydroxybutanoate, especially carboxylic acid ester of 4-oxo-4-(C₁-C₅-alkoxy)-2-butanol or of 4-oxo-4-(hydroxy-C₃-C₅-alkoxy)-2-butanol, obtainable according to the inventive production method or the inventive carboxylic acid ester of 3-hydroxybutanoate, especially carboxylic acid ester of 4-oxo-4-(C₁-C₅-alkoxy)-2-butanol or of 4-oxo-4-(hydroxy-C₃-C₅-alkoxy)-2-butanol, as defined hereinabove, respectively, and/or the mixture, obtainable according to the inventive production method or the inventive mixture as defined hereinabove, respectively, comprises a multitude of advantages and special features compared to the prior art:

As the applicant has surprisingly found out, the reaction product obtainable according to the inventive method or the inventive reaction product as defined hereinabove, respectively, and/or the carboxylic acid ester of 3-hydroxybutanoate, especially carboxylic acid ester of 4-oxo-4-(C₁-C₅-alkoxy)-2-butanol or of 4-oxo-4-(hydroxy-C₃-C₅-alkoxy)-2-butanol, obtainable according to the inventive production method or the inventive carboxylic acid ester of 3-hydroxybutanoate, especially carboxylic acid ester of 4-oxo-4-(C₁-C₅-alkoxy)-2-butanol or of 4-oxo-4-(hydroxy-C₃-C₅-alkoxy)-2-butanol, as defined hereinabove, respectively, and/or the mixture, obtainable according to the inventive production method or the inventive mixture as defined hereinabove, respectively, is suitable as a precursor or metabolite of 3-hydroxybutyric acid or its salts, since, on the one hand, it is converted physiologically, especially in the gastrointestinal tract, to 3-hydroxybutyric acid or its salts and, on the other hand, it simultaneously comprises a good physiological compatibility or tolerability, especially with regard to non-toxicity and acceptable organoleptic properties.

Moreover, the reaction product obtainable according to the inventive method or the inventive reaction product as defined hereinabove, respectively, and/or the carboxylic acid ester of 3-hydroxybutanoate, especially carboxylic acid ester of 4-oxo-4-(C₁-C₅-alkoxy)-2-butanol or of 4-oxo-4-(hydroxy-C₃-C₅-alkoxy)-2-butanol, obtainable according to the inventive production method or the inventive carboxylic acid ester of 3-hydroxybutanoate, especially carboxylic acid ester of 4-oxo-4-(C₁-C₅-alkoxy)-2-butanol or of 4-oxo-4-(hydroxy-C₃-C₅-alkoxy)-2-butanol, as defined hereinabove, respectively, and/or the mixture, obtainable according to the inventive production method or the inventive mixture as defined hereinabove, respectively, is easily accessible or available on a large scale on a synthetic basis, even on a commercial scale, and with the required pharmaceutical or pharmacological quality.

Additionally, the reaction product obtainable according to the inventive method or the inventive reaction product as defined hereinabove, respectively, and/or the carboxylic acid ester of 3-hydroxybutanoate, especially carboxylic acid ester of 4-oxo-4-(C₁-C₅-alkoxy)-2-butanol or of 4-oxo-4-(hydroxy-C₃-C₅-alkoxy)-2-butanol, obtainable according to the inventive production method or the inventive carboxylic acid ester of 3-hydroxybutanoate, especially carboxylic acid ester of 4-oxo-4-(C₁-C₅-alkoxy)-2-butanol or of 4-oxo-4-(hydroxy-C₃-C₅-alkoxy)-2-butanol, as defined hereinabove, respectively, and/or the mixture, obtainable according to the inventive production method or the inventive mixture as defined hereinabove, respectively, can, if necessary, be provided in enantiomerically pure or enantiomerically enriched form.

The reaction product obtainable according to the inventive method or the inventive reaction product as defined hereinabove, respectively, and/or the carboxylic acid ester of 3-hydroxybutanoate, especially carboxylic acid ester of 4-oxo-4-(C₁-C₅-alkoxy)-2-butanol or of 4-oxo-4-(hydroxy-C₃-C₅-alkoxy)-2-butanol, obtainable according to the inventive production method or the inventive carboxylic acid ester of 3-hydroxybutanoate, especially carboxylic acid ester of 4-oxo-4-(C₁-C₅-alkoxy)-2-butanol or of 4-oxo-4-(hydroxy-C₃-C₅-alkoxy)-2-butanol, as defined hereinabove, respectively, and/or the mixture, obtainable according to the inventive production method or the inventive mixture as defined hereinabove, respectively, thus represents an efficient pharmacological drug target in the context of keto-body therapy of the human or animal body.

In the following, the remaining aspects of the invention are explained in more detail.

A further subject-matter of the present invention—according to a third aspect of the present invention—is a pharmaceutical composition, especially a drug or medicament, which comprises a reaction product obtainable according to the inventive production method or the inventive reaction product as defined hereinabove, respectively, and/or a carboxylic acid ester of 3-hydroxybutanoate, especially carboxylic acid ester of 4-oxo-4-(C₁-C₅-alkoxy)-2-butanol or of 4-oxo-4-(hydroxy-C₃-C₅-alkoxy)-2-butanol, obtainable according to the inventive production method or the inventive carboxylic acid ester of 3-hydroxybutanoate, especially carboxylic acid ester of 4-oxo-4-(C₁-C₅-alkoxy)-2-butanol or of 4-oxo-4-(hydroxy-C₃-C₅-alkoxy)-2-butanol, as defined hereinabove, respectively, and/or a mixture, obtainable according to the inventive production method or the inventive mixture as defined hereinabove, respectively.

Especially, according to this aspect of the invention, the present invention relates to a pharmaceutical composition for the prophylactic and/or therapeutic treatment or for use in the prophylactic and/or therapeutic treatment of diseases of the human or animal body. This may especially concern diseases associated with a disorder of the energy metabolism, especially keto-body metabolism, such as especially craniocerebral trauma, stroke, hypoxia, cardiovascular diseases such as myocardial infarction, refeeding syndrome, anorexia, epilepsy, neurodegenerative diseases such as dementia, Alzheimer's disease, Parkinson's disease, multiple sclerosis and amyotrophic lateral sclerosis, fat metabolic diseases such as glucose transporter defect (GLUT1 defect), VL-FAOD and mitochondriopathies such as mitochondrial thiolase defect, Huntington's disease, cancers such as T-cell lymphomas, astrocytomas and glioblastomas, HIV, rheumatic diseases such as rheumatoid arthritis and arthritis urica, diseases of the gastrointestinal tract such as chronic inflammatory bowel diseases, especially ulcerative colitis and Crohn's disease, lyosomal storage diseases such as sphingolipidosis, especially Niemann-Pick disease, diabetes mellitus and effects or side-effects of chemotherapy.

Again, a further subject-matter of the present invention—according to a fourth aspect of the present invention—is a reaction product obtainable according to the inventive production method or the inventive reaction product as defined hereinabove, respectively, and/or a carboxylic acid ester of 3-hydroxybutanoate, especially carboxylic acid ester of 4-oxo-4-(C₁-C₅-alkoxy)-2-butanol or of 4-oxo-4-(hydroxy-C₃-C₅-alkoxy)-2-butanol, obtainable according to the inventive production method or the inventive carboxylic acid ester of 3-hydroxybutanoate, especially carboxylic acid ester of 4-oxo-4-(C₁-C₅-alkoxy)-2-butanol or of 4-oxo-4-(hydroxy-C₃-C₅-alkoxy)-2-butanol, as defined hereinabove, respectively, and/or a mixture, obtainable according to the inventive production method or the inventive mixture as defined hereinabove, respectively, for the prophylactic and/or therapeutic treatment or for use in the prophylactic and/or therapeutic treatment of diseases of the human or animal body, especially diseases associated with a disorder of the energy metabolism, especially keto-body metabolism, such as especially craniocerebral trauma, stroke, hypoxia, cardiovascular diseases such as myocardial infarction, refeeding syndrome, anorexia, epilepsy, neurodegenerative diseases such as dementia, Alzheimer's disease, Parkinson's disease, multiple sclerosis and amyotrophic lateral sclerosis, fat metabolic diseases such as glucose transporter defect (GLUT1 defect), VL-FAOD and mitochondriopathies such as mitochondrial thiolase defect, Huntington's disease, cancers such as T-cell lymphomas, astrocytomas and glioblastomas, HIV, rheumatic diseases such as rheumatoid arthritis and arthritis urica, diseases of the gastrointestinal tract such as chronic inflammatory bowel diseases, especially ulcerative colitis and Crohn's disease, lyosomal storage diseases such as sphingolipidosis, especially Niemann-Pick disease, diabetes mellitus and effects or side-effects of chemotherapy.

Likewise, a further subject-matter of the present invention—according to a fifth aspect of the present invention—is the use of a reaction product obtainable according to the inventive production method or the inventive reaction product as defined hereinabove, respectively, and/or a carboxylic acid ester of 3-hydroxybutanoate, especially carboxylic acid ester of 4-oxo-4-(C₁-C₅-alkoxy)-2-butanol or of 4-oxo-4-(hydroxy-C₃-C₅-alkoxy)-2-butanol, obtainable according to the inventive production method or the inventive carboxylic acid ester of 3-hydroxybutanoate, especially carboxylic acid ester of 4-oxo-4-(C₁-C₅-alkoxy)-2-butanol or of 4-oxo-4-(hydroxy-C₃-C₅-alkoxy)-2-butanol, as defined hereinabove, respectively, and/or a mixture, obtainable according to the inventive production method or the inventive mixture as defined hereinabove, respectively, for the prophylactic and/or therapeutic treatment or for producing a pharmaceutical for the prophylactic and/or therapeutic treatment of diseases of the human or animal body, especially diseases associated with a disorder of the energy metabolism, especially keto-body metabolism, such as especially craniocerebral trauma, stroke, hypoxia, cardiovascular diseases such as myocardial infarction, refeeding syndrome, anorexia, epilepsy, neurodegenerative diseases such as dementia, Alzheimer's disease, Parkinson's disease, multiple sclerosis and amyotrophic lateral sclerosis, fat metabolic diseases such as glucose transporter defect (GLUT1 defect), VL-FAOD and mitochondriopathies such as mitochondrial thiolase defect, Huntington's disease, cancers such as T-cell lymphomas, astrocytomas and glioblastomas, HIV, rheumatic diseases such as rheumatoid arthritis and arthritis urica, diseases of the gastrointestinal tract such as chronic inflammatory bowel diseases, especially ulcerative colitis and Crohn's disease, lyosomal storage diseases such as sphingolipidosis, especially Niemann-Pick disease, diabetes mellitus and effects or side-effects of chemotherapy.

Likewise, a further subject-matter of the present invention—according to a six th aspect of the present invention—is the use of a reaction product obtainable according to the inventive production method or the inventive reaction product as defined hereinabove, respectively, and/or a carboxylic acid ester of 3-hydroxybutanoate, especially carboxylic acid ester of 4-oxo-4-(C₁-C₅-alkoxy)-2-butanol or of 4-oxo-4-(hydroxy-C₃-C₅-alkoxy)-2-butanol, obtainable according to the inventive production method or the inventive carboxylic acid ester of 3-hydroxybutanoate, especially carboxylic acid ester of 4-oxo-4-(C₁-C₅-alkoxy)-2-butanol or of 4-oxo-4-(hydroxy-C₃-C₅-alkoxy)-2-butanol, as defined hereinabove, respectively, and/or a mixture, obtainable according to the inventive production method or the inventive mixture as defined hereinabove, respectively, for the prophylactic and/or therapeutic treatment or for producing a medicament for the prophylactic and/or therapeutic treatment of or for the application for catabolic metabolic states, such as hunger, diets or low-carbohydrate nutrition.

Likewise, a further subject-matter of the present invention—according to a seventh aspect of the present invention—is a food and/or a food product, which comprises a reaction product obtainable according to the inventive production method or the inventive reaction product as defined hereinabove, respectively, and/or carboxylic acid ester of 3-hydroxybutanoate, especially carboxylic acid ester of 4-oxo-4-(C₁-C₅-alkoxy)-2-butanol or of 4-oxo-4-(hydroxy-C₃-C₅-alkoxy)-2-butanol, obtainable according to the inventive production method or the inventive carboxylic acid ester of 3-hydroxybutanoate, especially carboxylic acid ester of 4-oxo-4-(C₁-C₅-alkoxy)-2-butanol or of 4-oxo-4-(hydroxy-C₃-C₅-alkoxy)-2-butanol, as defined hereinabove, respectively, and/or a mixture, obtainable according to the inventive production method or the inventive mixture as defined hereinabove, respectively.

According to a particular embodiment, the food and/or the food product may essentially be a dietary supplement, a functional food, a novel food, a food additive, a food supplement, a dietary food, a power snack, an appetite suppressant or a strength and/or endurance sport supplement.

Finally, yet another subject-matter of the present invention—according to an eighth aspect of the present invention—is the use of a reaction product obtainable according to the inventive production method and/or a carboxylic acid ester of 3-hydroxybutanoate, especially carboxylic acid ester of 4-oxo-4-(C₁-C₅-alkoxy)-2-butanol or of 4-oxo-4-(hydroxy-C₃-C₅-alkoxy)-2-butanol, obtainable according to the inventive production method or the inventive carboxylic acid ester of 3-hydroxybutanoate, especially carboxylic acid ester of 4-oxo-4-(C₁-C₅-alkoxy)-2-butanol or of 4-oxo-4-(hydroxy-C₃-C₅-alkoxy)-2-butanol, as defined hereinabove, respectively, and/or a mixture, obtainable according to the inventive production method in a food and/or a food product.

According to this aspect of the invention, the food and/or the food product may especially be a dietary supplement, a functional food, a novel food, a food additive, a food supplement, a dietary food, a power snack, an appetite suppressant or a strength and/or endurance sports supplement.

Further embodiments, modifications and variations of the present invention are readily recognizable or realizable by a person skilled in the art when reading the description, without leaving the scope of the present invention.

The present invention is illustrated by the following examples, which, however, are not intended to limit the present invention in any way, but are merely intended to explain the exemplary and non-limiting implementation and configuration of the present invention. The examples are further characterized with reference to the following description of the drawings and the drawings themselves. In this context, all features described and/or illustrated constitute, individually or in any combination, the subject-matter of the present invention, irrespective of their summary in the claims and their back-references.

It shows:

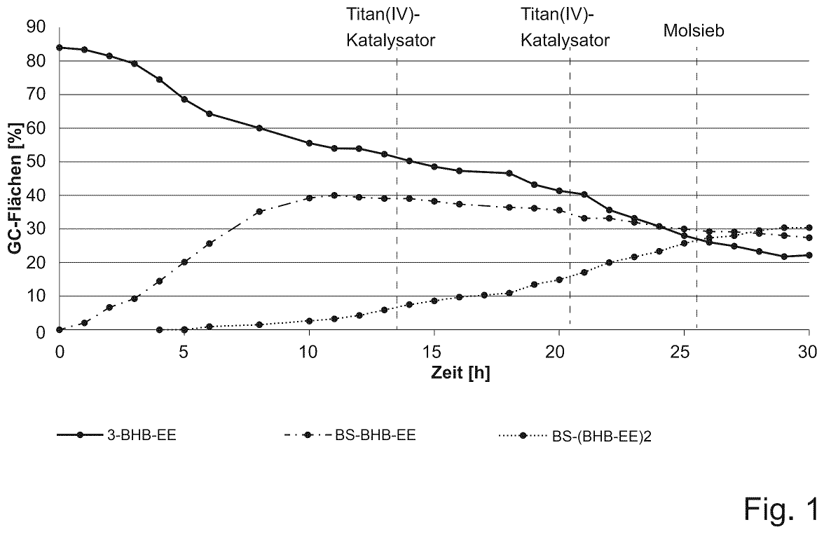

FIG. 1a plot of the conversion-time-course of the reaction of succinic acid anhydride with ethyl 3-hydroxybutanoate,

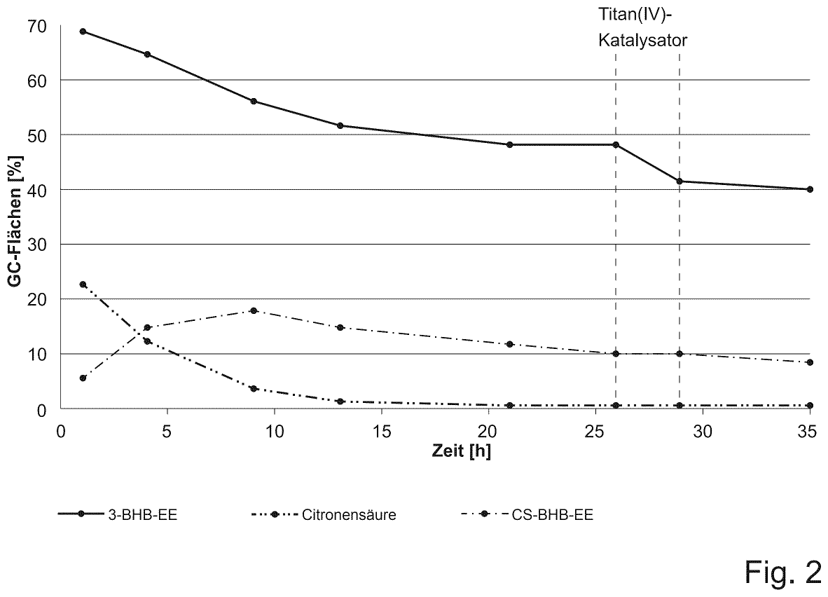

FIG. 2a plot of the conversion-time-course of the reaction of citric acid with ethyl 3-hydroxybutanoate,

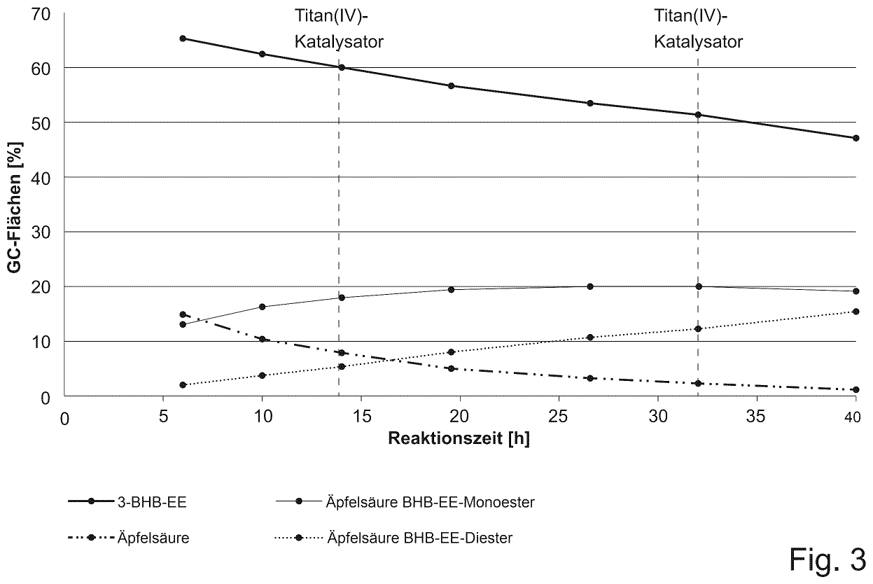

FIG. 3a plot of the conversion-time-course of the reaction of malic acid with ethyl 3-hydroxybutanoate,

FIG. 1 shows the conversion-time-course of the reaction of succinic anhydride with ethyl 3-hydroxybutanoate, wherein the X-axis shows the reaction time in hours [h] and the Y-axis shows the GC area analysis in percent [%]. Shown are the courses or amounts of the starting compound ethyl 3-hydroxybutanoate (3-BHB-EE) and the reaction products succinic acid monoester of ethyl 3-hydroxybutanoate (BS-BHB-EE) and succinic acid diester of ethyl 3-hydroxybutanoate (BS-(BHB-EE) 2). Furthermore, the times at which the titanium (IV)-catalyst is added and at which the molecular sieve is added for adsorptive removal of water are each marked.

During the entire reaction time, ethyl 3-hydroxybutanoate is converted. After 10 hours of reaction time, the conversion of succinic acid anhydride in the first reaction step to the succinic acid monoester of ethyl 3-hydroxybutanoate is already completed. The addition of the catalyst does not lead to a significant increase in conversion. The anew addition of catalyst leads to increased formation of the succinic acid diester of ethyl 3-hydroxybutanoate, with a slight decrease in the amount of succinic acid monoester of ethyl 3-hydroxybutanoate formed due to further conversion to the succinic acid diester of ethyl 3-hydroxybutanoate. The addition of the molecular sieve does not significantly increase the conversion. After 30 hours of reaction time, approximately equal amounts of succinic acid monoester and succinic acid diester of ethyl 3-hydroxybutanoate are present.

FIG. 2 shows the conversion-time-course of the reaction of citric acid with ethyl 3-hydroxybutanoate, wherein the X-axis shows the conversion time in hours [h] and the Y-axis shows the GC area analysis in percent [%]. Shown are the courses or amounts of the starting compound ethyl 3-hydroxybutanoate (3-BHB-EE) and citric acid and the reaction products citric acid monoester of ethyl 3-hydroxybutanoate (CS-BHB-EE), citric acid diester of ethyl 3-hydroxybutanoate (CS-(BHB-EE) 2) and citric acid triester of ethyl 3-hydroxybutanoate (CS-(BHB-EE) 3). Furthermore, the times at which the titanium (IV)-catalyst is added are each marked.

Throughout the reaction time, the ethyl 3-hydroxybutanoate is converted. At the beginning of the reaction, the citric acid mono ester and the citric acid diester of ethyl 3-hydroxybutanoate are formed significantly. After a reaction time of about 13 hours, the amount of citric acid monoester of ethyl 3-hydroxybutanoate decreases again because more citric acid monoester of ethyl 3-hydroxybutanoate is converted to the citric acid diester of ethyl 3-hydroxybutanoate than new citric acid monoester of ethyl 3-hydroxybutanoate is formed. After a reaction time of about 21 hours, the citric acid is completely reacted, so that no new citric acid monoester of ethyl 3-hydroxybutanoate is formed from this point on. With the first addition of the titanium (IV)-catalyst, a small increase in conversion is achieved with respect to the citric acid monoester and the citric acid diester of ethyl 3-hydroxybutanoate, but the second addition does not lead to a significant further increase in conversion.

FIG. 3 shows the conversion-time-course of the reaction of malic acid with ethyl 3-hydroxybutanoate, wherein the X-axis shows the reaction time in hours [h] and the Y-axis shows the GC area analysis in percent [%]. Shown are the courses or amounts of the starting compound ethyl 3-hydroxybutanoate (3-BHB-EE) and malic acid as well as the reaction products malic acid monoester of ethyl 3-hydroxybutanoate (malic acid BHB-EE monoester) and malic acid diester of ethyl 3-hydroxybutanoate (malic acid BHB-EE diester). Furthermore, the times at which the titanium (IV)-catalyst is added are each marked.

During the entire reaction time, the ethyl 3-hydroxybutanoate is converted. After 25 hours of reaction time, the conversion of the malic acid in the first reaction step to the malic acid monoester of ethyl 3-hydroxybutanoate is already completed. The additions of the catalyst each do not lead to any significant increases in conversion.

### EXAMPLES

Abbreviations used
3-BHB-EE=BHB-EE: 3-hydroxybutyric acid ethyl ester (=ethyl 3-hydroxybutanoate or 4-ethoxy-4-oxobutan-2-ol)
3-BHB=BHB: 3-hydroxybutyric acid
BS: succinic acid
CS: citric acid
   I. Examples of Production of a Particular Embodiment of the Method According to the Invention without the Use of a Catalyst

### I.1. Production of Tartaric Acid Esters of Ethyl 3-Hydroxybutanoate

In a 500-ml-multi-neck flask with dephlegmator (partial condenser) and distillation bridge, 132 g (R)/(S)-3-hydroxybutyric acid ethyl ester (3-BHB-EE=ethyl 3-hydroxybutanoate or 4-ethoxy-4-oxobutan-2-ol) (racemic) and 38 g tartaric acid are provided. The reaction mixture is reacted with stirring at 120° C. and under N₂ for 7 h, and the resulting reaction water is continuously removed by distillation. Subsequently, the excess 3-hydroxybutyric acid ethyl ester is distilled off under vacuum. The residue obtained is steam treated for 2 to 4 h in a high vacuum.

A mixture of tartaric acid monoester of ethyl 3-hydroxybutanoate [=mono-(4-ethoxy-4-oxo-butan-2-yl)-tartrate or tartaric acid mono-(4-ethoxy-4-oxo-butan-2-yl)-ester] and tartaric acid diester of ethyl 3-hydroxybutanoate [=bis-(4-ethoxy-4-oxo-butan-2-yl)-tartrate or tartaric acid di-(4-ethoxy-4-oxo-butan-2-yl)-ester] is obtained.

The tartaric acid monoester of ethyl 3-hydroxybutanoate may also be referred to synonymously as 4-[(4-ethoxy-4-oxobutan-2-yl)-oxy]-2,3-dihydroxy-4-oxobutanoic acid, while the tartaric acid diester of ethyl 3-hydroxybutanoate may also be referred to synonymously as 1,4-bis-(4-ethoxy-4-oxobutan-2-yl)-2,3-dihydroxybutanedioate) or also as bis-(4-ethoxy-4-oxobutan-2-yl)-2,3-hydroxysuccinate.

Characterization is performed by mass spectrometry (MS), gel permeation chromatography (GPC) and proton resonance spectroscopy (1H-NMR).

The reaction course is shown schematically below:

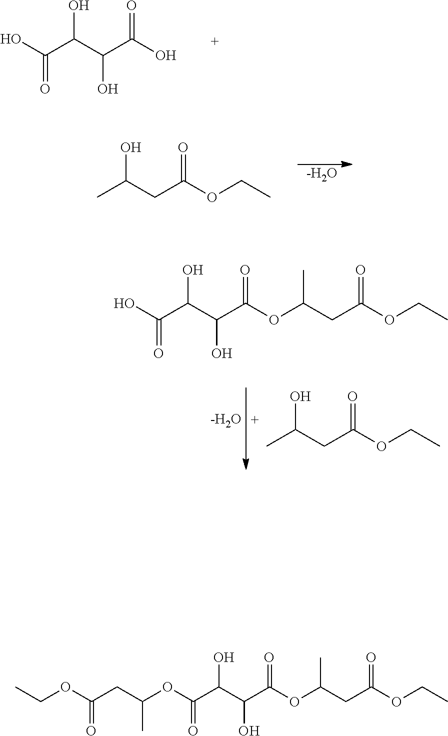

### I.2. Production of Lactic Acid Ester of Ethyl 3-Hydroxybutanoate

In a 500-ml-multi-neck flask with dephlegmator (partial condenser) and distillation bridge, 132 g (R)/(S)-3-hydroxybutyric acid ethyl ester (3-BHB-EE=ethyl 3-hydroxybutanoate or 4-ethoxy-4-oxobutan-2-ol) (racemic) and 57 g lactic acid are provided. The reaction mixture is reacted with stirring at 120° C. and under N₂ for 7 h and the resulting reaction water is continuously removed by distillation. Subsequently, the excess 3-hydroxybutyric acid ethyl ester is distilled off under vacuum. The residue obtained is steam treated for 2 to 4 h in a high vacuum.

The lactic acid monoester of ethyl 3-hydroxybutanoate [=mono-(4-ethoxy-4-oxo-butan-2-yl)-lactate or lactic acid mono-(4-ethoxy-4-oxo-butan-2-yl)-ester] is obtained. Characterization is performed by MS, GPC and 1H-NMR.

The reaction course is shown schematically below:

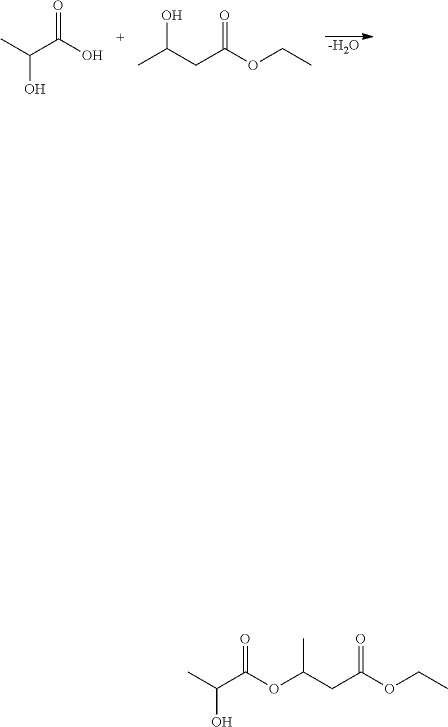

### I.3. Production of Succinic Acid Esters of Ethyl 3-Hydroxybutanoate

### Alternative 1: Use of Succinic Acid Anhydride

In a 500-ml-multi-neck flask with dephlegmator (partial condenser) and distillation bridge, 132 g (R)/(S)-3-hydroxybutyric acid ethyl ester (3-BHB-EE=ethyl 3-hydroxybutanoate or 4-ethoxy-4-oxobutan-2-ol) (racemic) and 24 g succinic acid anhydride are provided. The reaction mixture is reacted with stirring at 120° C. and under N₂ for 7 h and the resulting reaction water is continuously removed by distillation. Then the excess 3-hydroxybutyric acid ethyl ester is distilled off under vacuum. The residue obtained is steam treated for 2 to 4 h in a high vacuum.

A mixture of succinic acid monoester of ethyl 3-hydroxybutanoate [=mono-(4-ethoxy-4-oxo-butan-2-yl)-succinate or succinic acid mono-(4-ethoxy-4-oxo-butan-2-yl)-ester] and succinic acid diester of ethyl 3-hydroxybutanoate [=bis-(4-ethoxy-4-oxo-butan-2-yl)-succinate or succinic acid di-(4-ethoxy-4-oxo-butan-2-yl)-ester] is obtained. Characterization is performed by MS, GPC and 1H-NMR.

The succinic acid monoester of ethyl 3-hydroxybutanoate may also be referred to synonymously as 4-[(4-ethoxy-4-oxobutan-2-yl)-oxy]-4-oxobutanoic acid, while the succinic acid diester of ethyl 3-hydroxybutanoate may also be referred to synonymously as 1,4-bis-(4-ethoxy-4-oxobutan-2-yl)-butanedioate or also as bis-(4-ethoxy-4-oxobutan-2-yl)-succinate.

The reaction course is shown schematically below:

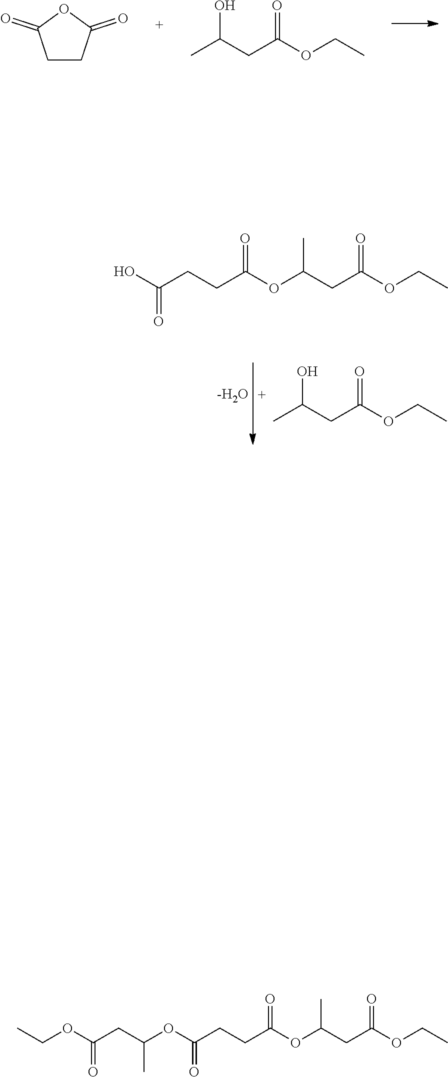

### Alternative 2: Use of the Free Succinic Acid

In a 500-ml-multi-neck flask with dephlegmator (partial condenser) and distillation bridge, 132 g (R)/(S)-3-hydroxybutyric acid ethyl ester (3-BHB-EE=ethyl 3-hydroxybutanoate or 4-ethoxy-4-oxobutan-2-ol) (racemic) and 33 g succinic acid are provided. The reaction mixture is reacted with stirring at 120° C. and under N₂ for 7 h, and the resulting reaction water is continuously removed by distillation. Subsequently, the excess 3-hydroxybutyric acid ethyl ester is distilled off under vacuum. The residue obtained is steam treated for 2 to 4 h in a high vacuum.

A mixture of succinic acid monoester of ethyl 3-hydroxybutanoate [=mono-(4-ethoxy-4-oxo-butan-2-yl)-succinate or succinic acid mono-(4-ethoxy-4-oxo-butan-2-yl)-ester] and succinic acid diester of ethyl 3-hydroxybutanoate [=bis-(4-ethoxy-4-oxo-butan-2-yl)-succinate or succinic acid di-(4-ethoxy-4-oxo-butan-2-yl)-ester] is obtained. Characterization is performed by MS, GPC and ¹H-NMR.

The succinic acid monoester of ethyl 3-hydroxybutanoate may also be referred to synonymously as 4-[(4-ethoxy-4-oxobutan-2-yl)-oxy]-4-oxobutanoic acid, while the succinic acid diester of ethyl 3-hydroxybutanoate may also be referred to synonymously as 1,4-bis-(4-ethoxy-4-oxobutan-2-yl)-butanedioate or also as bis-(4-ethoxy-4oxobutan-2-yl-)-succinate.

The reaction course is shown schematically below:

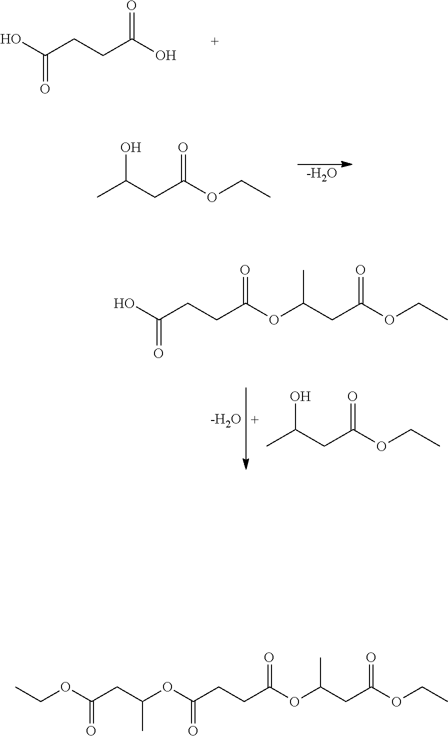

### I.4. Production of Citric Acid Esters of Ethyl 3-Hydroxybutanoate

In a 500-ml-multi-neck flask with dephlegmator (partial condenser) and distillation bridge, 132 g (R)/(S)-3-hydroxybutyric acid ethyl ester (3-BHB-EE=ethyl 3-hydroxybutanoate or 4-ethoxy-4-oxobutan-2-ol) (racemic) and 32 g citric acid are provided. The reaction mixture is reacted with stirring at 120° C. and under N₂ for 7 h and the resulting reaction water is continuously removed by distillation. Subsequently, the excess 3-hydroxybutyric acid ethyl ester is distilled off under vacuum. The residue obtained is steam treated for 2 to 4 h in a high vacuum.

A mixture of citric acid monoester of ethyl 3-hydroxybutanoate [=mono-(4-ethoxy-4-oxo-butan-2-yl)-citrate or citric acid mono-(4-ethoxy-4-oxo-butan-2-yl)-ester] and citric acid diester of ethyl 3-hydroxybutanoate [=bis-(4-ethoxy-4-oxo-butan-2-yl)-citrate or citric acid di-(4-ethoxy-4-oxo-butan-2-yl)-ester] and citric acid triester of ethyl 3-hydroxybutanoate [=tris-(4-ethoxy-4-oxo-butan-2-yl)-citrate or citric acid tri-(4-ethoxy-4-oxo-butan-2-yl)-ester]is obtained. Characterization is performed by MS, GPC and ¹H-NMR.

The citric acid monoester of ethyl 3-hydroxybutanoate may also be referred to synonymously as 2-{2-[(4-ethoxy-4-oxobutan-2-yl)-oxy]-2-oxoethyl}-2-hydroxybutanoic acid or also as 2-{2-[(4-ethoxy-4-oxobutan-2-yl)-oxy]-2-oxoethyl}-2-hydroxysuccinyl acid, while the citric acid diester of ethyl 3-hydroxy butanoate may also be referred to synonymously as 4-[(4-ethoxy-4-oxobutan-2-yl)-oxy]-2-{2-[(4-ethoxy-4-oxobutan-2-yl)-oxy]-2-oxoethyl}-2-hydroxy-4-oxobutanoic acid, and the citric acid triester of ethyl 3-hydroxybutanoate may also be referred to synonymously as 1,2,3-tris-(4-ethoxy-4-oxobutan-2yl)-2-hydroxypropane-1,2,3-tricarboxylate.

The reaction course is shown schematically below:

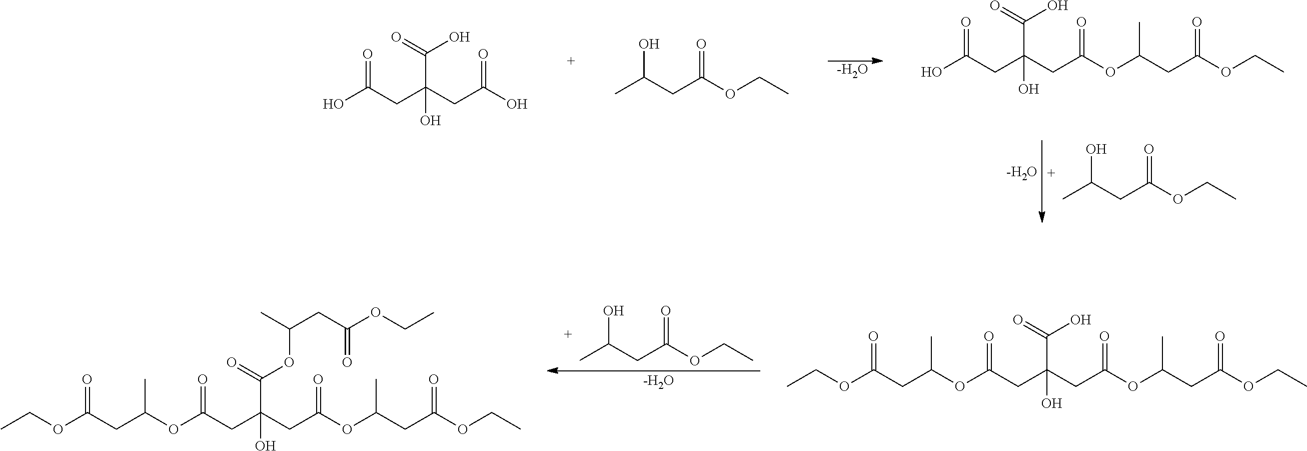

### I.5. Production of Maleic Esters of Ethyl 3-Hydroxybutanoate

### Alternative 1: Use of Maleic Acid Anhydride

In a 500-ml-multi-neck flask with dephlegmator (partial condenser) and distillation bridge, 132 g (R)/(S)-3-hydroxybutyric acid ethyl ester (3-BHB-EE=ethyl 3-hydroxybutanoate or 4-ethoxy-4-oxobutan-2-ol) (racemic) and 29 g maleic acid anhydride are provided. The reaction mixture is reacted with stirring at 120° C. and under N₂ for 7 h and the resulting reaction water is continuously removed by distillation. Subsequently, the excess 3-hydroxybutyric acid ethyl ester is distilled off under vacuum. The residue obtained is steam treated for 2 to 4 h in a high vacuum.

A mixture of maleic acid monoester of ethyl 3-hydroxybutanoate [=mono-(4-ethoxy-4-oxo-butan-2-yl)-maleate or maleic acid mono-(4-ethoxy-4-oxo-butan-2-yl)-ester] and maleic acid diester of ethyl 3-hydroxybutanoate [=bis-(4-ethoxy-4-oxo-butan-2-yl)-maleate or maleic acid di-(4-ethoxy-4-oxo-butan-2-yl)-ester] is obtained. Characterization is performed by MS, GPC and ¹H-NMR.

The reaction course is shown schematically below:

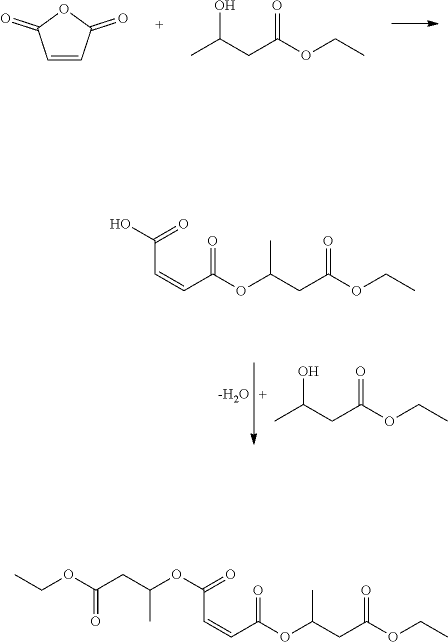

### Alternative 2: Use of the Free Maleic Acid

In a 500-ml-multi-neck flask with dephlegmator (partial condenser) and distillation bridge, 132 g (R)/(S)-3-hydroxybutyric acid ethyl ester (3-BHB-EE=ethyl 3-hydroxybutanoate or 4-ethoxy-4-oxobutan-2-ol) (racemic) and 33 g maleic acid are provided. The reaction mixture is reacted with stirring at 120° C. and under N₂ for 7 h and the resulting reaction water is continuously removed by distillation. Subsequently, the excess 3-hydroxybutyric acid ethyl ester is distilled off under vacuum. The residue obtained is steam treated for 2 to 4 h in a high vacuum.

A mixture of maleic acid monoester of ethyl 3-hydroxybutanoate [=mono-(4-ethoxy-4-oxo-butan-2-yl)-maleate or maleic acid mono-(4-ethoxy-4-oxo-butan-2-yl)-ester] and maleic acid diester of ethyl 3-hydroxybutanoate [=bis-(4-ethoxy-4-oxo-butan-2-yl)-maleate or maleic acid di-(4-ethoxy-4-oxo-butan-2-yl)-ester] is obtained. Characterization is performed by MS, GPC and 1H-NMR.

The reaction course is shown schematically below:

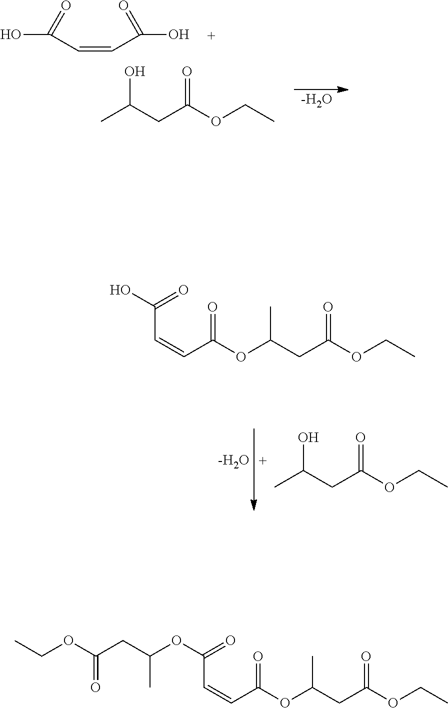

### I.6. Production of Fumaric Acid Esters of Ethyl 3-Hydroxybutanoate

In a 500-ml-multi-neck flask with dephlegmator (partial condenser) and distillation bridge, 132 g (R)/(S)-3-hydroxybutyric acid ethyl ester (3-BHB-EE=ethyl 3-hydroxybutanoate or 4-ethoxy-4-oxobutan-2-ol) (racemic) and 33 g fumaric acid are provided. The reaction mixture is reacted with stirring at 120° C. and under N₂ for 7 h and the resulting reaction water is continuously removed by distillation. Subsequently, the excess 3-hydroxybutyric acid ethyl ester is distilled off under vacuum. The residue obtained is steam treated for 2 to 4 h in a high vacuum.

A mixture of fumaric acid monoester of ethyl 3-hydroxybutanoate [=mono-(4-ethoxy-4-oxo-butan-2-yl)-fumarate or fumaric acid mono-(4-ethoxy-4-oxo-butan-2-yl)-ester] and fumaric acid diester of ethyl 3-hydroxybutanoate [=bis-(4-ethoxy-4-oxo-butan-2-yl)-fumarate or fumaric acid di-(4-ethoxy-4-oxo-butan-2-yl)-ester] is obtained. Characterization is performed by MS, GPC and 1H-NMR.

The reaction course is shown schematically below:

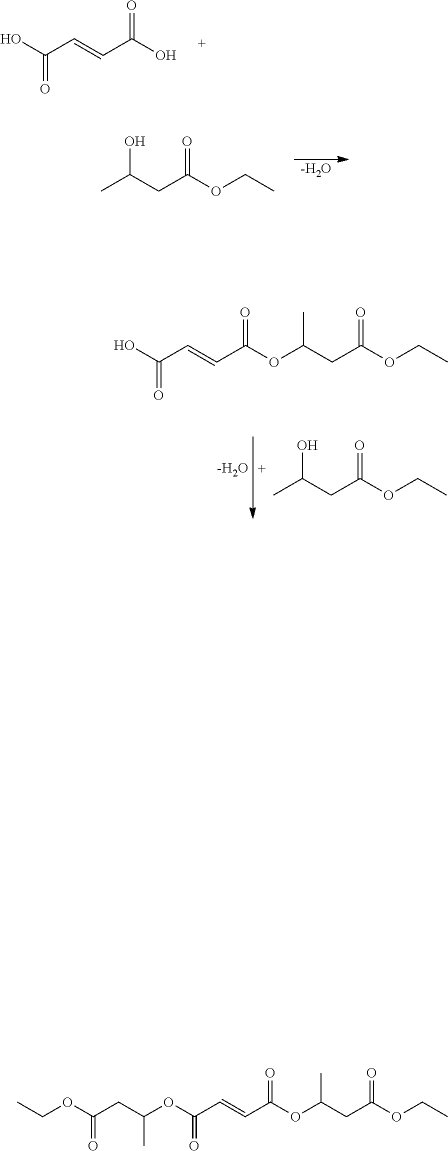

### I.7. Production of Malic Acid Esters of Ethyl 3-Hydroxybutanoate

In a 500-ml-multi-neck flask equipped with a dephlegmator (partial condenser) and distillation bridge, 132 g of (R)/(S)-3-hydroxybutyric acid ethyl ester (3-BHB-EE=ethyl 3-hydroxybutanoate or 4-ethoxy-4-oxobutan-2-ol) (racemic) and 33 g of malic acid (2-hydroxysuccinic acid) are provided. The reaction mixture is reacted with stirring at 120° C. and under N₂ for 7 h, and the resulting reaction water is continuously separated. Subsequently, the excess 3-hydroxybutyric acid ethyl ester is distilled off under vacuum. The residue obtained is steam treated for 2 to 4 h in a high vacuum.

A mixture of malic acid monoester of ethyl 3-hydroxybutanoate [=mono-(4-ethoxy-4-oxo-butan-2-yl)-malate or malic acid mono-(4-ethoxy-4-oxo-butan-2-yl)-ester] and malic acid diester of ethyl 3-hydroxybutanoate [=bis-(4-ethoxy-4-oxo-butan-2-yl)-malate or malic acid di-(4-ethoxy-4-oxo-butan-2-yl)-ester] is obtained. Characterization is performed by MS, GPC and 1H-NMR.

The malic acid monoester of ethyl 3-hydroxybutanoate may also be referred to synonymously as 4-[(4-ethoxy-4-oxobutan-2-yl-)-oxy]-2-hydroxy-4-oxobutanoic acid, while the malic diester of ethyl 3-hydroxybutanoate may also be referred to synonymously as 1,4-bis-(4-ethoxy-4-oxobutan-2-yl)-2-hydroxybutanedioate or also as bis-(4-ethoxy-4-oxobutan-2-yl)-2-hydroxysuccinate.

The reaction course is shown schematically below:

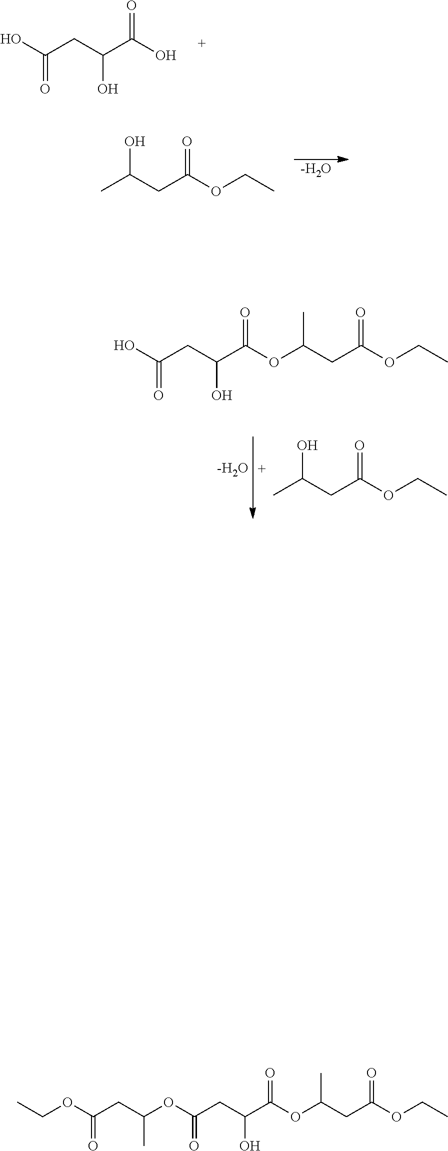

### I.8. Production of Adipic Acid Esters of Ethyl 3-Hydroxybutanoate

In a 500-ml-multi-neck flask with dephlegmator (partial condenser) and distillation bridge, 132 g (R)/(S)-3-hydroxybutyric acid ethyl ester (3-BHB-EE=ethyl 3-hydroxybutanoate or 4-ethoxy-4-oxobutan-2-ol) (racemic) and 37 g adipic acid are provided. The reaction mixture is reacted with stirring at 120° C. and under N₂ for 7 h, and the resulting reaction water is continuously removed by distillation. Subsequently, the excess 3-hydroxybutyric acid ethyl ester is distilled off under vacuum. The residue obtained is steam treated for 2 to 4 h in a high vacuum.

A mixture of adipic acid monoester of ethyl 3-hydroxybutanoate [=mono-(4-ethoxy-4-oxo-butan-2-yl)-adipate or adipic acid mono-(4-ethoxy-4-oxo-butan-2-yl)-ester] and adipic acid diester of ethyl 3-hydroxybutanoate [=bis-(4-ethoxy-4-oxo-butan-2-yl)-adipate or adipic acid di-(4-ethoxy-4-oxo-butan-2-yl)-ester] is obtained. Characterization is performed by MS, GPC and ¹H-NMR.

The reaction course is shown schematically below:

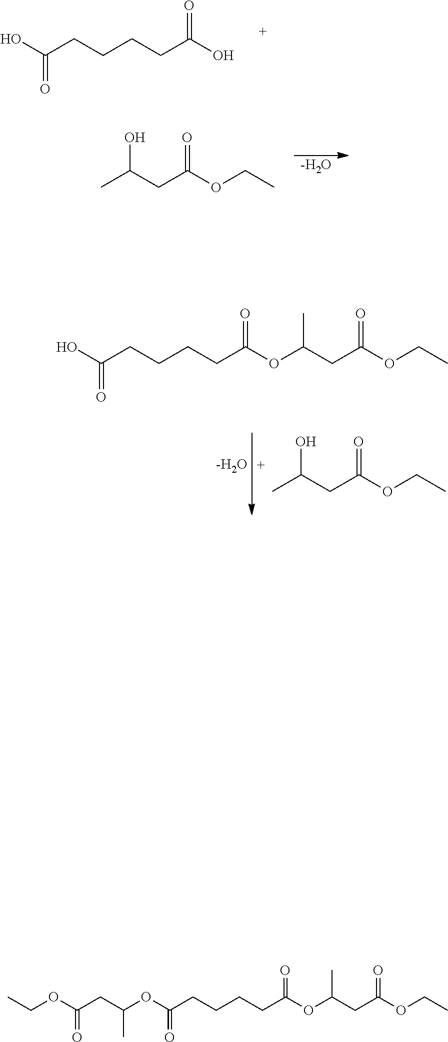

I.9. Production of Further Carboxylic Acid Esters of Ethyl 3-Hydroxybutanoate In addition, the above syntheses Nos. I.1, I.2, I.4 and I.6 to I.8 are each repeated, however, using the corresponding carboxylic acid anhydrides instead of the free carboxylic acids (i.e. in the case of Example I.1. tartaric acid anhydride instead of tartaric acid, in the case of Example I.2 lactic acid anhydride instead of lactic acid, in the case of Example I.4. citric acid anhydride instead of citric acid, in the case of Example I.6. fumaric acid anhydride instead of fumaric acid, in the case of Example I.7. malic acid anhydride instead of malic acid, in the case of Example I.8. adipic acid anhydride instead of adipic acid).

Comparable results are obtained. In this context, the corresponding carboxylic acids formed are not continuously removed during the reaction, but can continue to react with 3-hydroxybutyric acid ethyl ester. After the reaction is completed, excess 3-hydroxybutyric acid ethyl ester or excess carboxylic acid anhydride or free carboxylic acid (depending on the amount ratios of reactants used) is removed by distillation.

Furthermore, the previously mentioned examples No. I.1 to I.8 are repeated, however, case with other 3-hydroxybutanoates (namely each with 3-hydroxybutyric acid (hydroxybutyl) ester or 3-hydroxybutyric acid (hydroxypentyl) ester instead of 3-hydroxybutyric acid ethyl ester). 3-Hydroxybutyric acid (hydroxybutyl) ester is obtained by esterification of 3-hydroxybutyric acid with butanediol (e.g. with 1,3-butanediol), while 3-hydroxybutyric acid (hydroxypentyl) ester is obtained by esterification of 3-hydroxybutyric acid with pentanediol (e.g. with 1,3-pentanediol).

In a first series, the free carboxylic acids are used as reactants and, in a second series, the corresponding carboxylic acid anhydrides are used.

Comparable results are obtained. Purification and separation are performed in the same way.

Also, the previously mentioned examples are repeated, however, with the methyl and ethyl esters of the carboxylic acids. Comparable results are obtained. Purification and separation are carried out in the same way.

### II. Production Examples for an Alternative Particular Embodiment of the Method According to the Invention Using a Metal Catalyst

All chemical synthesis examples previously described in section I. are carried out again, however, with the addition of 1.6 g titanium tetrabutylate (titanium (IV)-catalyst). The titanium (IV)-catalyst is provided in the flask together with the other reactants. Subsequently, the course of the reaction corresponds to the examples described hereinabove. Comparable results are obtained. The catalyst is separated and recycled after the reaction.

### III. Production Examples for a Further Alternative Particular Embodiment of the Method According to the Invention Using an Enzyme Catalyst

All chemical synthesis examples previously described in section I. are carried out again, however, with the addition of an enzyme as a catalyst. Comparable results are obtained. The catalyst (i.e. the enzyme) is separated and recycled after the end of the reaction.

Some performed examples are selected and described below, with all other previously mentioned synthesis examples being performed analogously and providing comparable results.

### III.1. Production of Tartaric Acid Esters of Ethyl 3-Hydroxybutanoate

In a 500-ml-multi-neck flask with dephlegmator (partial condenser) and distillation bridge, 132 g (R)/(S)-3-hydroxybutyric acid ethyl ester (3-BHB-EE=ethyl 3-hydroxybutanoate or 4-ethoxy-4-oxobutan-2-ol) (racemic), 38 g tartaric acid and 1.7 g immobilized enzyme (CALB lipase on polymer support, derived from Candida antarctica, e.g. Novozym® 435 from Sigma-Aldrich or Merck or Lipozym® 435 from Strem Chemicals, Inc.) are provided. The reaction mixture is allowed to react with stirring at 70° C. and under vacuum (<500 mbar) for 7 h. The enzyme is then filtered off and the excess 3-hydroxybutyric acid ethyl ester is distilled off under vacuum. The residue obtained is steam treated for 2 to 4 h in a high vacuum.

A mixture of tartaric acid monoester of ethyl 3-hydroxybutanoate [=mono-(4-ethoxy-4-oxo-butan-2-yl)-tartrate or tartaric acid mono-(4-ethoxy-4-oxo-butan-2-yl)-ester] and tartaric acid diester of ethyl 3-hydroxybutanoate [=bis-(4-ethoxy-4-oxo-butan-2-yl)-tartrate or tartaric acid di-(4-ethoxy-4-oxo-butan-2-yl)-ester] is obtained. Characterization is performed by MS, GPC and ¹H-NMR.

### III. 2. Production of Lactic Acid Ester of Ethyl 3-Hydroxybutanoate

In a 500-ml-multi-neck flask with dephlegmator (partial condenser) and distillation bridge, 132 g (R)/(S)-3-hydroxybutyric acid ethyl ester (3-BHB-EE=ethyl 3-hydroxybutanoate or 4-ethoxy-4-oxobutan-2-ol) (racemic), 57 g lactic acid and 1.8 g immobilized enzyme (CALB lipase on polymer support, derived from Candida antarctica, e.g. Novozym® 435 from Sigma-Aldrich or Merck or Lipozym® 435 from Strem Chemicals, Inc.) are provided. The reaction mixture is allowed to react with stirring at 70° C. and under vacuum (<500 mbar) for 7 h. The enzyme is then filtered off and the excess 3-hydroxybutyric acid ethyl ester is distilled off under vacuum. The residue obtained is steam treated for 2 to 4 h in a high vacuum.

The lactic acid monoester of ethyl 3-hydroxybutanoate [=mono-(4-ethoxy-4-oxo-butan-2-yl)-lactate or lactic acid mono-(4-ethoxy-4-oxo-butan-2-yl)-ester] is obtained. Characterization is performed by MS, GPC and ¹H-NMR.

### III.3. Production of Succinic Acid Esters of Ethyl 3-Hydroxybutanoate

In a 500-ml-multi-neck flask with dephlegmator (partial condenser) and distillation bridge, 132 g (R)/(S)-3-hydroxybutyric acid ethyl ester (3-BHB-EE=ethyl 3-hydroxybutanoate or 4-ethoxy-4-oxobutan-2-ol) (racemic), 33 g succinic acid and 1.6 g immobilized enzyme (CALB lipase on polymer support, derived from Candida antarctica, e.g. Novozym® 435 from Sigma-Aldrich or Merck or Lipozym® 435 from Strem Chemicals, Inc.) are provided. The reaction mixture is allowed to react with stirring at 70° C. and under vacuum (<500 mbar) for 7 h. The enzyme is then filtered off and the excess 3-hydroxybutyric acid ethyl ester is distilled off under vacuum. The residue obtained is steam treated for 2 to 4 h in a high vacuum.

A mixture of succinic acid monoester of ethyl 3-hydroxybutanoate [=mono-(4-ethoxy-4-oxo-butan-2-yl)-succinate or succinic acid mono-(4-ethoxy-4-oxo-butan-2-yl)-ester] and succinic acid diester of ethyl 3-hydroxybutanoate [=bis-(4-ethoxy-4-oxo-butan-2-yl)-succinate or succinic acid di-(4-ethoxy-4-oxo-butan-2-yl)-ester] is obtained. Characterization is performed by MS, GPC and ¹H-NMR-.

### III.4. Production of Citric Acid Esters of Ethyl 3-Hydroxybutanoate

In a 500-ml-multi-neck flask with dephlegmator (partial condenser) and distillation bridge, 132 g (R)/(S)-3-hydroxybutyric acid ethyl ester (3-BHB-EE=ethyl 3-hydroxybutanoate or 4-ethoxy-4-oxobutan-2-ol) (racemic), 32 g citric acid and 1.8 g immobilized enzyme (CALB lipase on polymer support, derived from Candida antarctica, e.g. Novozym® 435 from Sigma-Aldrich or Merck or Lipozym® 435 from Strem Chemicals, Inc.) are provided. The reaction mixture is allowed to react with stirring at 70° C. and under vacuum (<500 mbar) for 7 h. The enzyme is then filtered off and the excess 3-hydroxybutyric acid ethyl ester is distilled off under vacuum. The residue obtained is steam treated for 2 to 4 h in a high vacuum.

A mixture of citric acid monoester of ethyl 3-hydroxybutanoate [=mono-(4-ethoxy-4-oxo-butan-2-yl)-citrate or citric acid mono-(4-ethoxy-4-oxo-butan-2-yl)-ester] and citric acid diester of ethyl 3-hydroxybutanoate [=bis-(4-ethoxy-4-oxo-butan-2-yl)-citrate or citric acid di-(4-ethoxy-4-oxo-butan-2-yl)-ester] and citric acid triester of ethyl 3-hydroxybutanoate [=tris-(4-ethoxy-4-oxo-butan-2-yl)-citrate or citric acid tri-(4-ethoxy-4-oxo-butan-2-yl)-ester] of ethyl 3-hydroxybutanoate is obtained. Characterization is performed by MS, GPC and ¹H-NMR.

### III.5. Production of Maleic Esters of Ethyl 3-Hydroxybutanoate

In a 500-ml-multi-neck flask with dephlegmator (partial condenser) and distillation bridge, 132 g (R)/(S)-3-hydroxybutyric acid ethyl ester (3-BHB-EE=ethyl 3-hydroxybutanoate or 4-ethoxy-4-oxobutan-2-ol) (racemic), 33 g maleic acid and 1.6 g immobilized enzyme (CALB lipase on polymer support, derived from Candida antarctica, e.g. Novozym® 435 from Sigma-Aldrich or Merck or Lipozym® 435 from Strem Chemicals, Inc.) are provided. The reaction mixture is allowed to react with stirring at 70° C. and under vacuum (<500 mbar) for 7 h. The enzyme is then filtered off and the excess 3-hydroxybutyric acid ethyl ester is distilled off under vacuum. The residue obtained is steam treated for 2 to 4 h in a high vacuum.

A mixture of maleic acid monoester of ethyl 3-hydroxybutanoate [=mono-(4-ethoxy-4-oxo-butan-2-yl)-maleate or maleic acid mono-(4-ethoxy-4-oxo-butan-2-yl)-ester] and maleic acid diester of ethyl 3-hydroxybutanoate [=bis-(4-ethoxy-4-oxo-butan-2-yl)-maleate or maleic acid di-(4-ethoxy-4-oxo-butan-2-yl)-ester] is obtained. Characterization is performed by MS, GPC and ¹H-NMR.

### III.6. Production of Malic Acid Esters of Ethyl 3-Hydroxybutanoate

In a 500-ml-multi-neck flask with dephlegmator (partial condenser) and distillation bridge, 132 g (R)/(S)-3-hydroxybutyric acid ethyl ester (3-BHB-EE=ethyl 3-hydroxybutanoate or 4-ethoxy-4-oxobutan-2-ol) (racemic), 33 g malic acid (2-hydroxysuccinic acid) and 1.6 g immobilized enzyme (CALB lipase on polymer support, derived from Candida antarctica, e.g. Novozym® 435 from Sigma-Aldrich or Merck or Lipozym® 435 from Strem Chemicals, Inc.) are provided. The reaction mixture is allowed to react with stirring at 70° C. and under vacuum (<500 mbar) for 7 h. The enzyme is then filtered off and the excess 3-hydroxybutyric acid ethyl ester is distilled off under vacuum. The residue obtained is steam treated for 2 to 4 h in a high vacuum.

A mixture of malic acid monoester of ethyl 3-hydroxybutanoate [=mono-(4-ethoxy-4-oxo-butan-2-yl)-malate or malic acid mono-(4-ethoxy-4-oxo-butan-2-yl)-ester] and malic acid diester of ethyl 3-hydroxybutanoate [=bis-(4-ethoxy-4-oxo-butan-2-yl)-malate or malic acid di-(4-ethoxy-4-oxo-butan-2-yl)-ester] is obtained. Characterization is performed by MS, GPC and ¹H-NMR.

### III.7. Production of Adipic Acid Esters of Ethyl 3-Hydroxybutanoate

In a 500-ml-multi-neck flask with dephlegmator (partial condenser) and distillation bridge, 132 g (R)/(S)-3-hydroxybutyric acid ethyl ester (3-BHB-EE=ethyl 3-hydroxybutanoate or 4-ethoxy-4-oxobutan-2-ol) (racemic) and 37 g adipic acid and 1.7 g immobilized enzyme (CALB lipase on polymer support, derived from Candida antarctica, e.g. e.g. Novozym® 435 from Sigma-Aldrich or Merck or Lipozym® 435 from Strem Chemicals, Inc.) are provided. The reaction mixture is allowed to react with stirring at 70° C. and under vacuum (<500 mbar) for 7 h. The enzyme is then filtered off and the excess 3-hydroxybutyric acid ethyl ester is distilled off under vacuum. The residue obtained is steam treated for 2 to 4 h in a high vacuum.

A mixture of adipic acid monoester of ethyl 3-hydroxybutanoate [=mono-(4-ethoxy-4-oxo-butan-2-yl)-adipate or adipic acid mono-(4-ethoxy-4-oxo-butan-2-yl)-ester] and adipic acid diester of ethyl 3-hydroxybutanoate [=bis-(4-ethoxy-4-oxo-butan-2-yl)-adipate or adipic acid di-(4-ethoxy-4-oxo-butan-2-yl)-ester] is obtained. Characterization is performed by MS, GPC and ¹H-NMR-.

### III.8. Production of Further Carboxylic Acid Esters of Ethyl 3-Hydroxybutanoate

Furthermore, the previously mentioned production examples are repeated, however, with other 3-hydroxybutanoates (namely 3-hydroxybutyric acid (hydroxybutyl) ester and 3-hydroxybutyric acid (hydroxypentyl) ester instead of 3-hydroxybutyric acid ethyl ester, respectively). Comparable results are obtained. Purification and separation are carried out in the same way.

Also, the previously mentioned production examples are repeated, however, with the methyl and ethyl esters of the carboxylic acids. Comparable results are obtained. Purification and separation are carried out in the same way.

### IV. Kinetics and Structural Analysis

In addition, the kinetics of the inventive method is analyzed. For this purpose, the conversion-time-courses of the reactions of succinic acid anhydride with ethyl 3-hydroxybutanoate (3-hydroxybutyric acid ethyl ester), citric acid with ethyl 3-hydroxybutanoate (3-hydroxybutyric acid ethyl ester) and malic acid with ethyl 3-hydroxybutanoate (3-hydroxybutyric acid ethyl ester) are determined.

IV.1. Reaction of Succinic Anhydride with Ethyl 3-Hydroxybutanoate

In a 500-ml-multi-neck flask with dephlegmator (partial condenser) and distillation bridge, 525 g (R)/(S)-3-hydroxybutyric acid ethyl ester (3-BHB-EE=ethyl 3-hydroxybutanoate or 4-ethoxy-4-oxobutan-2-ol) (racemic) and 133 g succinic acid anhydride are reacted for 13 h at 130° C. and 70 mbar. The mixture is then divided in half.

To the first half, 100° C. three drops of titanium tetrabutylate (titanium (IV)-catalyst) are added to the reaction mixture and the reaction is stirred for another 17 h at 130° C., gradually reducing the pressure to 40 mbar. After 7 h of the reaction time, fresh titanium tetrabutylate is added and after another 6 h 22 g of molecular sieve (Molsieve) 3 Å is added to remove the reaction water. After the reaction time is completed, the reaction mixture is filtered.

During the entire reaction time, samples are taken at regular intervals and the composition is analyzed. The resulting conversion-time-course is shown in FIG. 1.

From the conversion-time-course, it can be seen that after 10 h reaction time, the conversion of succinic acid anhydride in the first reaction step to BS-E-BHB monoester (succinic monoester of ethyl 3-hydroxybutanoate) is already complete. The addition of the catalyst does not lead to a significant increase in conversion.

The second half of the mixture is purified for further analysis. First, the excess 3-BHB-EE is removed and the residue is purified by column chromatography. For structure identification of the succinic acid esters of ethyl 3-hydroxybutanoate, the individual esters are each isolated by column chromatographic purification (i.e. the monoester and the diester are isolated). For this purpose, the 3-BHB-EE excess is removed and the product mixture is subsequently columnized over silica gel. An ethyl acetate/cyclohexane mixture in the ratio 2/1 with 0.5% triethylamine is used as the running medium. Subsequently, the two fractions A and B as well as a non-fractionated sample are analyzed by GC (gas chromatography); the GC area analyses are summarized in Table 1.

TABLE 1
GC area analysis [%] of the reaction of succinic
acid anhydride with ethyl 3-hydroxybutanoate

| | before | | |
|---|---|---|---|
| | fractionation | fraction A | fraction B |
| 3-BHB-EE | 0.9 | — | — |
| 3-BHB | 0.1 | — | — |
| succinic acid | 1.5 | — | — |
| 3-BHB derivatives * | 2.1 | 0.11 | — |
| BS-(BHB-EE) monoester | 77.7 | 97.6 | 0.23 |
| BS-(BHB-EE)-Diester | 14.7 | — | 94.39 |
| by-products ** | 1.6 | 1.44 | 4.56 |
| unknown | 1.6 | 0.84 | 0.82 |

* various 3-BHB derivatives (e.g. oligomers such as dimers, trimers and tetramers, etc.)
** various oligomeric and polycondensed BS-(BHB-EE)-derivatives as well as BS-derivatives (e.g. dimers, trimers, di- and polyesters, etc., respectively)

For further characterization of fractions A and B, ¹H-NMR and ¹³C-NMR spectra and 2D-NMR spectra (COSY, HSQC, HMBC) are measured. The spectra show (just like GC analysis) that fraction A comprises the succinic acid monoester of ethyl 3-hydroxybutanoate and fraction B comprises the succinic acid diester of ethyl 3-hydroxybutanoate.

The structure for the succinic acid monoester of ethyl 3-hydroxybutanoate (from fraction A) is shown below, and the chemical shifts uniquely assignable by the NMR spectra for the ¹H-NMR signals (1), and the ¹³C-NMR signals (2) are labeled:

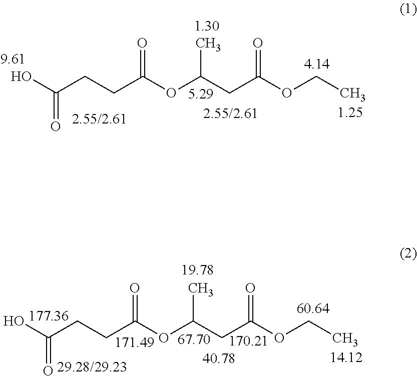 [SMILES: CCOC(=O)CC(C)OC(=O)CCC(=O)O ; CCOC(=O)CC(C)OC(=O)CCC(=O)O]

The structure for succinic diester of ethyl 3-hydroxybutanoate (from fraction B) is shown below, and the chemical shifts uniquely assignable by the NMR spectra for the ¹H-NMR signals (3) and the ¹³C-NMR signals (4) are labeled:

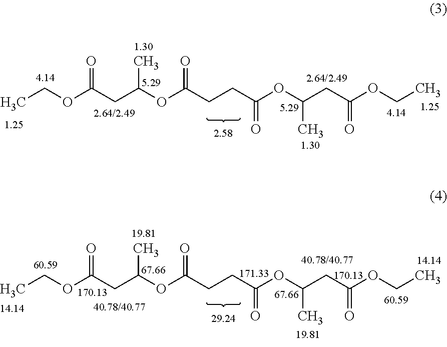

Subsequently, fractions A and B are each further purified (e.g. chromatographically). In the case of fraction A, pure succinic acid monoester of ethyl 3-hydroxybutanoate (purity>99%) is obtained and in the case of fraction B, pure succinic acid diester of ethyl 3-hydroxybutanoate (purity>99%) is obtained. A 1:1 mixture of monoester/diester is also prepared from a portion of the purified esters.

IV.2 Reaction of Citric Acid with Ethyl 3-Hydroxybutanoate

In a 500-ml-multi-neck flask with dephlegmator (partial condenser) and distillation bridge, 599 g (R)/(S)-3-hydroxybutyric acid ethyl ester (3-BHB-EE=ethyl 3-hydroxybutanoate or 4-ethoxy-4-oxobutan-2-ol) (racemic) and 146 g citric acid are reacted at 130° C. and 70 mbar. After 26 h reaction time, 67 mg titanium tetrabutylate (titanium (IV)-catalyst) is added to the reaction mixture and after another 3 h reaction time, another 43 mg titanium tetrabutylate (titanium (IV)-catalyst) is added to the reaction. The reaction mixture is then allowed to react for a further 6 h.

During the entire reaction time, samples are taken at regular intervals and the composition is analyzed. The resulting conversion-time-course is shown in FIG. 2.

From the conversion-time-course it can be seen that initially the citric acid monoester and the citric acid diester of ethyl 3-hydroxybutanoate are significantly formed. A small increase in conversion is obtained with the first addition of the titanium (IV)-catalyst, however, the second addition does not lead to a significant further increase in conversion.

The product mixture is purified for further analysis. First, the excess of 3-BHB-EE is removed and the residue is then purified by column chromatography. For structure identification of the citric acid esters of ethyl 3-hdroxybutanoate, the individual esters are isolated each by column chromatographic purification. For this purpose, the product mixture is chromatographed over a chromatographic column filled with silica gel, and an ethyl acetate/cyclohexane mixture in the ratio 5/3 with 0.7% glacial acetic acid is used as the running medium. Subsequently, both fractions A and B, as well as a non-fractionated sample before and after removal of the excess 3-BHB-EE, are analyzed by GC (gas chromatography); the GC area analyses are summarized in Table 2.

TABLE 2
GC area analysis [%] of the reaction of
citric acid with ethyl 3-hydroxy butanoate

| | before | before | | |
|---|---|---|---|---|
| | fractio- | fractio- | | |
| | nation; | nation; | frac- | frac- |
| | before 3-BHB- | after 3-BHB- | tion | tion |
| | EE removal | EE removal | A | B |
| 3-BHB-EE | 39.8 | 0.3 | 1 | 0.1 |
| 3-BHB | 0.1 | 0.1 | — | — |
| 3-BHB derivatives * | 14.9 | 8.7 | 1.5 | 5.2 |
| CS-(BHB-EE) monoester | 8.5 | 15.3 | — | 3.5 |
| CS-(BHB-EE)-diester | 22.2 | 41.8 | 6 | 65.1 |
| CS-(BHB-EE)-triester | 10.7 | 23.1 | 71 | 21.4 |
| by-products ** | 3.3 | 9.5 | 12 | 3.3 |
| unknown | 0.6 | 1.3 | 8.4 | 1.4 |

* various 3-BHB derivatives (e.g. oligomers such as dimers, trimers, and tetramers; deprotonated derivatives such as acetoacetates, etc.).
** various oligomeric and polycondensed CS-(BHB-EE)-derivatives as well as CS-derivatives (e.g. each dimers, trimers, di- and polyesters etc.)

The GC analysis shows that mainly the di- and tri-esters of citric acid of 3-hydroxybutanoate are formed; i.e. the citric acid monoester of 3-hydroxybutanoate is almost completely further converted to the corresponding diester. Fraction A comprises predominantly the citric acid diester of 3-hydroxybutanoate and fraction B comprises predominantly the citric acid diester of 3-hydroxybutanoate.

For further characterization of fraction A, ¹H-NMR and ¹³C-NMR spectra as well as 2D-NMR spectra (COSY, HSQC, HMBC) are measured. The spectra show (just like the GC analysis) that fraction A has the citric acid triester of ethyl 3-hydroxybutanoate. The three different stereocenters result in 4 diastereomeric compounds and correspondingly many signals within each group. The isomer mixture itself has a high purity.

The structure for the citric acid triester of ethyl 3-hydroxybutanoate (from fraction A) is shown below, and the chemical shifts uniquely assignable by the NMR spectra for the ¹H-NMR signals (5) and the ¹³C-NMR signals (6) are labeled:

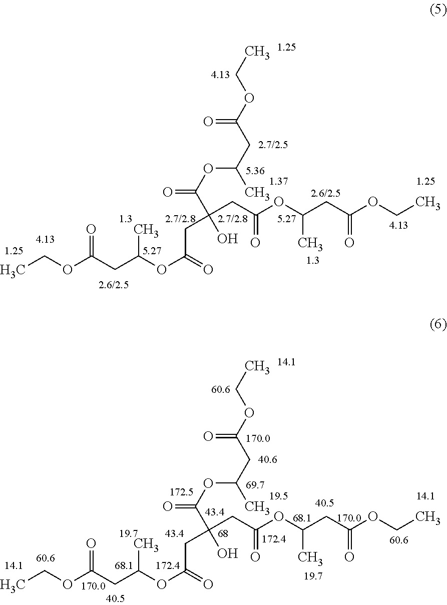

Subsequently, fractions A and B are each further purified (e.g. chromatographically). In the case of fraction A, pure citric acid triester of ethyl 3-hydroxybutanoate (purity>99%) is obtained and in the case of fraction B, a mixture of citric acid di- and citric acid monoester of ethyl 3-hydroxybutanoate (purity>99%) is obtained. A 1:1:1 mixture of monoester/diester/triester is also prepared from a portion of the purified esters.

IV.3. Reaction of Malic Acid with Ethyl 3-Hydroxybutanoate

In a 500-ml-multi-neck flask with dephlegmator (partial condenser) and distillation bridge, 476 g of (R)/(S)-3-hydroxybutyric acid ethyl ester (3-BHB-EE=ethyl 3-hydroxybutanoate or 4-ethoxy-4-oxobutan-2-ol) (racemic) and 121 g of malic acid are reacted for 10 h at 120° C. and 70 mbar. Subsequently, 100 g are removed and the remainder is further reacted. After 4 h of reaction, the reaction mixture is cooled to 100° C. and 100 mg of titanium tetrabutylate (titanium (IV)-catalyst) is added to the reaction mixture. Then the reaction is reheated to 120° C. and stirred for another 24 h at constant temperature and 60 mbar, and fresh titanium tetrabutylate (titanium (IV)-catalyst) is added after 18 h reaction time.

During the entire reaction time, samples are taken at regular intervals and the composition is analyzed. The resulting conversion-time-course is shown in FIG. 3.

From the conversion-time-course, it is evident that the addition of the titanium (IV)-catalyst does not lead to a significant increase in conversion.

The 100 g of reaction mixture removed is purified for further analysis. For this purpose, the excess of 3-BHB-EE is first removed and the radical is then purified by column chromatography. For this purpose, the product mixture is chromatographed over a chromatography column filled with silica gel, and an ethyl acetate/cyclohexane mixture in the ratio 2/1 with 1% glacial acetic acid is used as the running medium. Subsequently, both fractions A and B as well as a non-fractionated sample before and after removal of the excess 3-BHB-EE are analyzed by GC (gas chromatography); the GC area analyses are summarized in Table 3.

TABLE 3
GC area analysis [%] of the reaction of
malic acid with ethyl 3-hydroxybutanoate

| | before | fraction | fraction |
|---|---|---|---|
| | fractionation | A | B |
| 3-BHB-EE | 22.9 | 0.8 | 0.8 |
| 3-BHB | 0.5 | — | — |
| succinic acid | 0.6 | — | 1.1 |
| malic acid | 13.5 | — | 0.5 |
| 3-BHB derivatives * | 5.6 | 3.0 | — |
| BS-(BHB-EE) monoester | 0.9 | 5.1 | — |
| malic acid (BHB-EE) monoester | 33.3 | 0.2 | 88.9 |
| malic acid dimer | 2.4 | 3.4 | — |
| malic acid (BHB-EE) diester | 11.3 | 73.1 | 4.6 |
| by-products ** | 7.2 | 11.9 | 1.2 |
| unknown | 1.9 | 2.5 | 1.2 |

* various 3-BHB derivatives (e.g. oligomers such as dimers, trimers and tetramers, etc.)
** various oligomeric and polycondensed malic acid (BHB-EE) derivatives as well as malic acid derivatives (e.g. each dimers, trimers, di- and polyesters etc.)

For further characterization of fractions A and B, ¹H-NMR and ¹³C-NMR spectra as well as 2D-NMR spectra (COSY, HSQC, HMBC) are measured. The spectra show (just like the GC analysis) that fraction A comprises the malic acid diester of ethyl 3-hydroxybutanoate and fraction B comprises the malic acid monoester of ethyl 3-hydroxybutanoate.

The structure for the malic diester of ethyl 3-hydroxybutanoate (from fraction A) is shown below, and the chemical shifts uniquely assignable by the NMR spectra for the ¹H-NMR signals (7) and the ¹³C-NMR signals (8) are labeled:

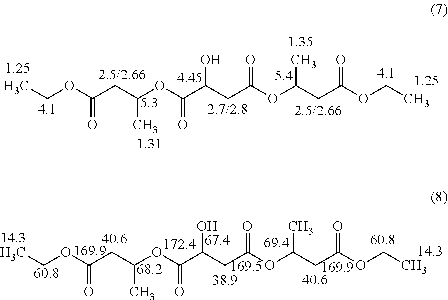

The structure for malic diester of ethyl 3-hydroxybutanoate (from fraction B) is shown below, and the chemical shifts uniquely assignable by the NMR spectra for the ¹H-NMR signals (9) and the ¹³C-NMR signals (10) are labeled:

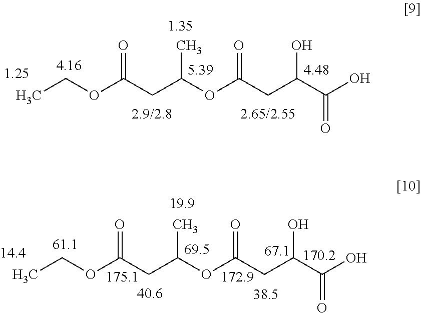 [SMILES: CCOC(=O)CC(C)OC(=O)CC(O)C(=O)O ; CCOC(=O)CC(C)OC(=O)CC(O)C(=O)O]

Subsequently, fractions A and B are each further purified (e.g. chromatographically). In the case of fraction A, pure malic acid diester of ethyl 3-hydroxybutanoate (purity>99%) is obtained and in the case of fraction B, pure malic acid monoester of ethyl 3-hydroxybutanoate (purity>99%) is obtained. A 1:1 mixture of monoester/diester is also prepared from a portion of the purified esters.

### V. Physiological Application Tests: In-Vitro Digestion Tests

### V.1. Digestion Experiments (Splitting or Cleavage Tests) of Inventive Carboxylic Acid Esters of 3-Hydroxybutanoate

By means of cleavage experiments it is shown that carboxylic acid esters of 3-hydroxybutanoate produced according to the invention or mixtures thereof (cf. previously described experiments according to I., II. and III.), including reaction by-products such as dimers, etc., can be cleaved in the human gastrointestinal tract.

In each case, purified reaction products obtained by the inventive method are used as starting mixtures (i.e. tartaric acid ester of 3-hydroxybutanoate, lactic acid ester of 3-hydroxybutanoate, succinic acid ester of 3-hydroxybutanoate, citric acid ester of 3-hydroxybutanoate, maleic acid ester of 3-hydroxybutanoate, fumaric acid ester of 3-hydroxybutanoate, malic acid ester of 3-hydroxybutanoate and adipic acid ester of 3-hydroxybutanoate).

For the cleavage experiments under near-body conditions two media are investigated:
FaSSGF, which simulates the stomach
FaSSIF, which simulates the intestinal tract

Both media are from the company Biorelevant®, Ltd. in Great Britain. In addition, in some experiments porcine pancreas is added (Panzytrat® 40,000, Fa. Allergan).

The results of the cleavage experiments in a FaSSGF or FaSSIF medium with Panzytrat® and without Panzytrat® (both 35° C., 24 h) show that the samples hydrolyze under FaSSGF conditions with Panzytrat® and without Panzytrat®; this is mainly due to the low pH value (pH=1.6) of the medium. Under FaSSIF conditions, a lower conversion using Panzytrat® takes place.

In the cleavage experiments of, for example, the citric acid triesters of 3-hydroxy butanoates, it can be seen that the cleavage of 3-hydroxybutanoate proceeds in a cascade (i.e. the citric acid triester becomes the citric acid diester, the citric acid diester becomes the citric acid monoester and the citric acid monoester finally becomes the free citric acid, wherein in each step the corresponding 3-hydroxybutanoate is released, which can subsequently be further cleaved leading to the free 3-hydroxybutyric acid and ethanol).

Accordingly, the cleavage experiments of dicarboxylic acid esters of 3-hydroxybutanoates also proceed in a cascade. Thus, a retardation effect is present overall.

### V.2. Further Digestion Experiments (Cleavage Experiments) of Inventive Carboxylic Acids of 3-Hydroxybutanoate

Cleavage Experiments with Pancreatin

2 g of a carboxylic acid ester of 3-hydroxybutanoate or a mixture thereof (e.g. mixture of corresponding mono- and di- or mono-, di- and tricarboxylic acid esters of 3-hydroxybutanoate) prepared as described hereinabove are dissolved in 50 g of water and 0.5 g (1% by weight) of pancreatin is added. The pancreatin is used in the form of the commercially available product Panzytrat® 40,000 from Allergan. The whole mixture is stirred on a hot plate at 50° C.; the course of the reaction is determined and followed by continuous recording of the acid number over time. The acid number increases over the observation period (cleavage of the carboxylic acid esters of 3-hydroxybutanoate to the free 3-hydroxybutyric acid). The conversion-time-course of the aqueous cleavage of the carboxylic acid esters of 3-hydroxybutyric acid by pancreatin according to the invention, including increase of the acid number over time, proves the desired decomposition of the reactant mixture to the free carboxylic acid and the free 3-hydroxybutyric acid. This is confirmed by appropriate analytics. The experiment proves that the starting mixture according to the invention is a suitable physiological precursor for 3-hydroxybutyric acid or its esters (3-hydroxybutanoates) for the corresponding keto-body therapies.

The test is repeated and verified on the basis of the individual esters in pure form. Comparable results are obtained in each case, i.e. the carboxylic acid esters of 3-hydroxybutanote are each cleaved by pancreatin to the free carboxylic acid and 3-hydroxybutanoate or 3-hydroxybutyric acid.

### V.3. Conclusion

The previously described cleavage experiments prove that the carboxylic acid esters of 3-hydroxybutanoate are efficient precursors or metabolites of free hydroxybutyric acid or its esters (here: ethyl esters, hydroxybutyl esters and hydroxypentyl esters), especially with regard to their intended effect, which are present in physiologically tolerable or physiologically compatible form. Likewise, metabolically utilizable or convertible carboxylic acids occurring in the natural metabolism (e.g. citrate cycle) or their derivatives, especially salts, are formed (e.g. citric acid or citrates, malic acid or malates, tartaric acid or tartrates, etc.).

### VI. Further Testing (Organoleptic and Toxicity)

Further experiments and test series are carried out with respect to organoleptic and toxicity of the carboxylic acid esters of 3-hydroxybutanoate according to the invention. These show that the carboxylic acid esters of 3-hydroxybutanoate according to the invention are organoleptically acceptable and compatible, and especially exhibit significantly improved organoleptic properties compared with pure 3-hydroxybutyric acid and its salts and esters, and also do not exhibit any toxicity contrary to the application.

1. A method for producing carboxylic acid esters of 3-hydroxybutanoate,
wherein at least one 3-hydroxybutanoate of general formula (I) 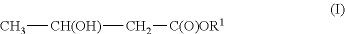
wherein, in general formula (I), the radical R¹ is selected among C₁-C₅-alkyl and hydroxy-C₃-C₅-alkyl,
is reacted, in an esterification reaction, with at least one carboxylic acid (II) selected from the group consisting of succinic acid, tartaric acid, lactic acid, citric acid, malic acid, adipic acid, fumaric acid and maleic acid and salts, anhydrides and esters thereof as well as combinations or mixtures thereof;
wherein the reaction is carried out in the absence of any solvent, and
wherein the reaction is carried out either in the absence of any catalyst or else in the presence of an enzyme as a catalyst;
so that, as a reaction product (III), one or more carboxylic acid esters of 3-hydroxybutanoate are obtained.

2. The method according to claim 1,
wherein the carboxylic acid (II) is used in the form of the free carboxylic acid, in the form of a salt of the carboxylic acid, in the form of an ester of the carboxylic acid or in the form of the carboxylic acid anhydride.

3. The method according to claim 1,
wherein the 3-hydroxybutanoate of the general formula (I), based on the carboxyl groups of the carboxylic acid (II), is used in molar amounts in a range of from equimolar amount up to a molar excess of 200 mol-%.

4. The method according to claim 1,
wherein the 3-hydroxybutanoate of the general formula (I) and the carboxylic acid (II) are used in a molar ratio of 3-hydroxybutanoate of the general formula (I)/carboxylic acid (II) in a range of from 1:1 to 10:1.

5. The method according to claim 1,
wherein, during the reaction of 3-hydroxybutanoate of the general formula (I) with a carboxylic acid (II) in the form of the free acid, water is formed simultaneously, wherein the water is continuously withdrawn from reaction.

6. The method according to claim 1,
wherein, during the reaction of 3-hydroxybutanoate of the general formula (I) with a carboxylic acid (II) in the form of the anhydride, 1 mol of the corresponding free carboxylic acid (II) is formed per 1 mol of the anhydride used, wherein the resulting free carboxylic acid (II) is reacted with 3-hydroxybutanoate of the general formula (I) or, after the reaction has taken place, is removed and recycled.

7. The method according to claim 1,
wherein hydroxyl groups and carboxyl groups still present in the reaction product after the reaction has been performed are at least partially esterified.

8. The method according to claim 1,
wherein the reaction is followed by an at least partial functionalization of hydroxyl groups and carboxyl groups still present.

9. A method of treating a human or an animal suffering from a disease of the human or animal body,
wherein the method comprises the step of administering to said human or animal a therapeutically efficient amount of at least one carboxylic acid ester of 3-hydroxybutanoate corresponding to general formula (IIIb) 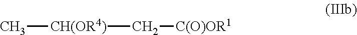
wherein, in the general formula (IIIb),
the radical R¹ is selected among C₁-C₅-alkyl and hydroxy-C₃-C₅-alkyl, and
the radical R⁴ is selected among one or more of the following radicals: 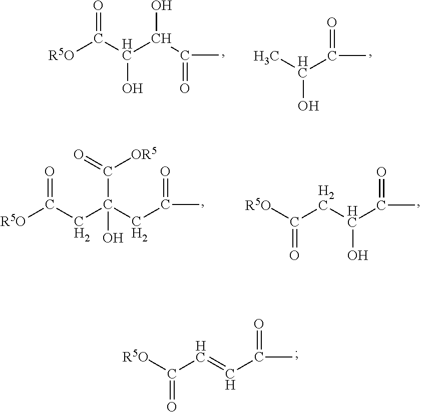
wherein, in said radicals, the radical R⁵ represents hydrogen or a radical —CH(CH₃)—CH₂—C(O)OR¹ with R¹ being selected among C₁-C₅-alkyl and hydroxy-C₃-C₅-alkyl.

10. The method according to claim 9,
wherein the disease is selected among diseases associated with a disorder of human or animal energy metabolism.

11. The method according to claim 9,
wherein the disease is selected among diseases associated with a disorder of human or animal keto-body metabolism.

12. The method according to claim 9,
wherein the disease is selected among craniocerebral trauma, stroke, hypoxia, cardiovascular diseases, myocardial infarction, refeeding syndrome, anorexia, epilepsy, neurodegenerative diseases, dementia, Alzheimer's disease, Parkinson's disease, multiple sclerosis, amyotrophic lateral sclerosis, fat metabolic diseases, glucose transporter defect, GLUT1 defect, very long-chain acyl-CoA dehydrogenase deficiency, mitochondriopathies, mitochondrial thiolase defect, Huntington's disease, cancers, T-cell lymphomas, astrocytomas, glioblastomas, HIV, rheumatic diseases, rheumatoid arthritis, arthritis urica, diseases of the gastrointestinal tract, chronic inflammatory bowel diseases, ulcerative colitis, Crohn's disease, lyosomal storage diseases, sphingolipidosis, Niemann-Pick disease, diabetes mellitus and chemotherapy-caused side-effects.
